SEQUENCE LISTING (8 sequences)

SEQ ID NO: 1 - protein, 9 aa, Artificial Sequence
  CDR Sequence
FSLTSYAVH

SEQ ID NO: 2 - protein, 16 aa, Artificial Sequence
  CDR Sequence
VIWAGGITHYNSALMS

SEQ ID NO: 3 - protein, 9 aa, Artificial Sequence
  CDR Sequence
ARGNWAFDY

SEQ ID NO: 4 - protein, 10 aa, Artificial Sequence
  CDR Sequence
SARSSVSYIH

SEQ ID NO: 5 - protein, 7 aa, Artificial Sequence
  CDR Sequence
DTSKLAS

SEQ ID NO: 6 - protein, 9 aa, Artificial Sequence
  CDR Sequence
FQGSGYPYT

SEQ ID NO: 7 - protein, 330 aa, Homo sapiens [sequence omitted]

SEQ ID NO: 8 - protein, 105 aa, Homo sapiens
PREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2018/0298084 A1
(43) Pub. Date: Oct. 18, 2018

(54) BACTERICIDAL MONOCLONAL ANTIBODY TARGETING KLEBSIELLA PNEUMONIAE

(71) Applicant: ARSANIS BIOSCIENCES GMBH, Vienna (AT)

(72) Inventors: Eszter NAGY, Wien (AT); Gábor NAGY, Sopron (HU); Valeria SZIJÁRTO, Vienna (AT); Luis Miguel GUACHALLA, Vienna (AT); Irina MIRKINA, Vienna (AT); Zehra VISRAM, Vienna (AT)

(73) Assignee: ARSANIS BIOSCIENCES GMBH, Vienna (AT)

(21) Appl. No.: 15/768,274

(22) PCT Filed: Oct. 14, 2016
(86) PCT No.: PCT/EP2016/074728

§ 371 Date: Apr. 13, 2018

(30) Foreign Application Priority Data
Oct. 16, 2015 (EP) 15190136.0

(51) Int. Cl. C07K 16/12; G01N 33/569; A61K 39/40; A61K 9/00; A61P 31/04

(52) U.S. Cl. CPC C07K 16/1228; G01N 33/56916; A61K 39/40; A61K 9/006; A61K 2039/545; A61P 31/04; A61K 2300/00; G01N 2333/26; A61K 9/0019

(57) ABSTRACT

A human or humanized monoclonal IgG antibody (mAb) specifically recognizing the D-galactan-II antigen of Klebsiella pneumoniae O1 which is characterized by a bactericidal CDC activity, its method of production, medical and diagnostic use.

The invention refers to a bactericidal monoclonal antibody (mAb) which is a human IgG1 antibody specifically recognizing D-galactan-II (galactan II, D-gal II, gal-II) within the LPS side chain of Klebsiella pneumoniae serotype O1, and its medical use.

### BACKGROUND OF THE INVENTION

Klebsiella pneumoniae is a nosocomial opportunistic pathogen responsible for urinary tract infections, pneumonia, and septicaemia, which cause significant morbidity and mortality. The susceptible patients often have impaired immune functions unable to cope with invasive infections caused by this commensal enterobacterium.

Even more alarming is that multi-drug resistant (MDR) strains have recently emerged and spread globally, against which therapeutic options are limited. Monoclonal antibodies may represent a novel therapeuticic approach. Nevertheless, molecular targets accessible on the surface of K. pneumonia are very limited given the bulky capsular polysaccharide that shields most surface antigens. On the other hand the readily accessible capsular polysaccharide shows high structural and hence antigenical variability that renders it non-attractive for broad spectrum antibacterial approaches.

The other major non-proteinaceous surface antigen is LPS that shows less variability than the capsular antigen. In K. pneumonia there are less than 10 O-serogroups distinguished based on the structure of the LPS O-side chains. The most common serotype is O1, which was reported to be expressed by more than one third of all K. pneumoniae isolates (1; 2). Held et al. described a murine galactan-II-specific murine IgG2b (Mab Ru.O1) which was capable of inducing complement-dependent opsonophagocytic killing, but lacked complement mediated killing, thus was not bactericidal in the absence of phagocytes (1).

An internal study including recent MDR strains isolated at different geographical locations confirmed the highest prevalence of O1 isolates (FIG. 1).

Naturally occurring antibodies consist of two heavy chains and two light chains. Within IgG, the fragment antigen binding (Fab) region contains the paratope, and can exert direct effects through binding interactions with antigen. Besides, the Fc region interacts with a variety of accessory molecules to mediate indirect effector functions such as antibody-dependent cellular cytotoxicity (ADCC), antibody-dependent cellular phagocytosis (ADCP) also known as opsonophagocytosis (OPK) and complement-dependent cytotoxicity (CDC). These latter two Fc mediated effector functions are especially important against infectious diseases where cellular and complement mediated responses are important for efficient pathogen clearance.

In complement-dependent cytotoxicity (CDC), the C1q binds the antibody and this binding triggers the complement cascade which leads to the formation of the membrane attack complex (C5b to C9) at the surface of the target cell, as a result of the classical pathway complement activation. The level of CDC effector function is typically high for human IgG1 and IgG3, low for IgG2, and null for IgG4, but mainly depends on the type of target cell and antigen.

In the most clinically prevalent K. pneumoniae serotype, D-galactan II provides the epitope that defines the O1 antigen, and is characterized by the D-gal II repeat unit structure: [-3)-α-D-Galp-(1-3)-β-D-Galp-(1−].

Its presence is responsible for the resistance of the bacteria to complement-mediated killing in the host. K. pneumoniae mutants that only produce D-galactan I are therefore serum-sensitive (13).

Phagocytes are cells which are able to absorb, engulf (phagocytose) and digest particles, microbes or dead cells. Professional phagocytes include neutrophil granulocytes, monocytes, macrophages, dendritic cells and mast cells.

The detailed biochemical structure of K. pneumoniae O1 antigen was described earlier by two independent groups (11; 12). Polyclonal and murine monoclonal IgG2a antibodies against the K. pneumoniae O1 antigen were raised and characterized by the Trautmann group as described above (1-3). Based on their experimental data the therapeutic use of anti-O1 mAbs was suggested.

Antibodies against LPS O-antigens, in particular against the O1 antigen (galactan-II) were developed and tested previously by others. It was shown that such antibodies induce opsonophagocytotic killing (OPK) (1) and afford protection in murine models of Klebsiella infections (2). Based on the opsonizing potential, the use of mAbs against galactan-II was implied as promising antibacterial strategy (2; 3).

Hsieh et al. described D-galactan II as an immunodominant antigen in O1 LPS and its implications in vaccine design (14).

There is prior art for the detailed structural analysis of the O1 antigen as well as monoclonal antibodies against this structure. Such prior art antibodies were selected for its opsonisation function, i.e. efficacy would rely on phagocyte function of the infected host. K. pneumonia, as an opportunistic pathogen, however, tends to infect immunocompromised individuals (see above) whose phagocytic activity may be severely compromised.

Kubota et al. describe engineered therapeutic antibodies with improved effector functions, such as antibody-dependent cytotoxicity and complement-dependent cytotoxicity (15).

Immunocompromised patients are unable to develop a normal immune response resulting in weaker/impaired immune system (immunodeficiency). Immunodeficiencies can be primary (when genetic defects affect immune cells) or secondary (when factors affect a host with an intrinsically normal immune system resulting in acquired immunodeficiency) and they can result from disorders of antibodies, lymphocytes, phagocytes, the complement system or combination of these factors.

As K. pneumoniae typically causes outbreaks in nosocomial settings, patients present at the same clinical ward, sharing medical equipment or personnel with a K. pneumoniae infected patient are at high risk of contracting infection.

### SUMMARY OF THE INVENTION

It is the objective of the present invention to provide an improved antibody that can be used for treating a human subject for immunoprophylaxis and immunotherapy, in particular for treating immunocompromised patients.

The object is solved by the subject of the present invention.

According to the invention, there is provided a humanized or human monoclonal IgG antibody (mAb) specifically recognizing D-galactan-II of Klebsiella pneumoniae serotype O1, specifically a D-galactan-II epitope within the LPS, or the D-galactan-II antigen, which antibody is characterized by a bactericidal CDC activity. Specifically, the antibody comprises a Fc region comprising a C1q binding site, characterized by a bactericidal CDC activity. In particular, the antibody as described herein is a mAb specifically recognizing the D-galactan-II antigen of Klebsiella pneumoniae O1 comprising a human constant region comprising a C1q binding site, characterized by a bactericidal CDC activity. Specifically, the antibody comprises the structure of an IgG1 or IgG3 antibody, preferably comprising the Fc of human IgG1 or IgG3. Specifically, the antibody is an IgG1 or IgG3 antibody.

As used herein, complement dependent cytotoxicity (CDC) of an antibody is the reaction wherein one or more complement protein components recognize bound antibody on a target cell and subsequently cause lysis of the target cell.

Specifically, the antibody is characterized by the CDC activity to complement-mediated direct killing of the antigen bearing bacterium in the circulation, as determined in serum, e.g. by a standard CDC assay. In particular, the antibody has bactericidal CDC activity, if there is a significant increase in the percentage of cytolysis as compared to a control. The cytotoxic activity related to CDC is preferably measured as the absolute percentage increase, which is preferably higher than 5%, more preferably higher than 10%, even more preferred higher than 20%.

It was surprising that a variety of monoclonal human antibodies of the IgG1 type, each with different antigen binding sites and different CDR sequences, were capable of directly killing the bacteria by CDC activity despite the natural resistance of K. pneumoniae serotype O1 to serum killing. The bactericidal activity is particularly relevant when treating patients with a phagocytic defect, or immunocompromised patients.

Specifically, the antibody is a humanized (including e.g., chimeric mAbs such as human/mouse mAbs, or other humanized mAbs such as those obtained upon CDR grafting to a human IgG1 framework), or human mAb.

According to a specific embodiment, the antibody comprises

A

the antigen-binding site characterized by the following CDR sequences:

a) CDR1 consisting of the amino acid sequence of SEQ ID 1; and

b) CDR2 consisting of the amino acid sequence of SEQ ID 2; and

c) CDR3 consisting of the amino acid sequence of SEQ ID 3; and

d) CDR4 consisting of the amino acid sequence of SEQ ID 4; and

e) CDR5 consisting of the amino acid sequence of SEQ ID 5; and

f) CDR6 consisting of the amino acid sequence of SEQ ID 6;

or

B

a functional variant of the antigen-binding site as defined in A, wherein the functional variant comprises at least one point mutation in any one or more of the CDR sequences, and further wherein

i. the functional variant has a specificity to bind the gal-II epitope; and/or

ii. the functional variant is a human, humanized, including e.g. human/mouse chimeric, or an affinity matured variant antigen-binding site.

Specifically, the CDR1-3 sequences are incorporated into a variable domain of an antibody heavy chain (VH domain), and the CDR4-6 sequences are incorporated into a variable domain of an antibody light chain (VL domain), employing human VH and VL framework sequences, or framework sequences which are at least 60% identical to human framework sequences, preferably at least any of 70%, 80%, or 90% identical. According to a specific example, the antibody is a humanized antibody comprising VH which incorporates the CDR1, CDR2, and CDR3 sequences within VH framework sequences, and VL which incorporates the CDR4, CDR5, and CDR6 sequences within VL framework sequences, wherein the framework sequences originate from a human IgG, in particular a human IgG1, or wherein at least one functional variant of a framework sequence is used which does not change the antigen-binding specificity of the variable domains, and which has at least 60% identity to the respective VH or VL framework sequence, preferably at least any of 70%, 80%, or 90% identity.

Specifically, the antigen-binding site as defined in A is of any of the exemplary antibodies designated as 8E9, G2-27, or G2-33 and described herein. Each of these antibodies is characterized by the same antigen-binding site as defined by the CDR1-6 sequences, but the antibodies differ in the framework or constant regions. Human-mouse chimeric mAb 8E9 comprises the human IgG1 constant heavy and kappa constant light chain regions, thus is of the human IgG1 type, as well as the humanized G2-27 and G2-33 which were obtained upon further humanization of the mAb 8E9.

The invention also refers to variants of such antibodies. For the purpose of providing variants, any of the 8E9, G2-27, or G2-33 antibodies are herein referred to as parent antibodies, and their CDR sequences are herein referred to as parent CDR sequences. The antibodies comprising functional variant of the antigen-binding site of the parent antibodies or any of its respective CDR sequences, are specifically understood as functional variant antibodies, and their variant CDR sequences are herein referred to as functionally active CDR variant sequences.

Unless indicated otherwise, reference is made to the CDR sequences as numbered according to Kabat, i.e. as determined according to Kabat nomenclature (see Kabat et al., Sequences of Proteins of Immunological Interest, 5th Ed. Public Health Service, U.S. Department of Health and Human Services. (1991)). It is well understood that the invention and the scope of the claims shall also encompass the same antibodies and CDR, yet with a different numbering and designated CDR region, where CDR regions are defined according to the IMGT system (The international ImMunoGeneTics, Lefranc et al., 1999, Nucleic Acids Res. 27: 209-212).

In particular, the variant antibodies binding to the target antigen and being cytotoxic as characterized by the CDC activity are considered functionally active. It is feasible that also variant VH or VL domains of a parent antibody, e.g. with modifications in the respective FR or CDR sequences may be used, which are functionally active, e.g. binding to the same epitope (i.e. the gal-II epitope) or comprising the same binding site or having the same binding characteristics as the parent antibody. It is also feasible that some of the FR or CDR sequences of the antibodies described herein may be exchanged by those of other antibodies. Specific variants may comprise
a combination of VH and VL domains obtained by shuffling the domains of the parent antibodies, or
a combination of HC and LC obtained by shuffling the heavy and light chains of the parent antibodies.

For example, a functional antibody variant may comprise a VH domain of a first parent antibody and a VL domain of another parent antibody. According to another example, the functional antibody variant may comprise a HC domain of a first parent antibody and a LC of another parent antibody.

The functional variant, also referred to as “functionally active” variant, can be a functionally active CDR variant which comprises at least one point mutation in the parent CDR sequence, and comprises or consists of the amino acid sequence that has at least 60% sequence identity with the parent CDR sequence, preferably at least 70%, at least 80%, at least 90% sequence identity.

A specific variant is e.g., a humanized variant of the parent antibody, wherein the parent CDR sequences are incorporated into human or humanized framework sequences, wherein optionally 1, 2, 3, or 4 amino acid residues of each of the parent CDR sequences may be further mutated by introducing point mutations to improve the stability, specificity and affinity of the parent or humanized antibody.

Specifically the antibody comprises a functionally active CDR variant of any of the CDR sequences of a parent antibody, wherein the functionally active CDR variant comprises at least one of

a) 1, 2, or 3 point mutations in the parent CDR sequence; and/or

b) 1 or 2 point mutations in any of the four C-terminal or four N-terminal, or four centric amino acid positions of the parent CDR sequence; and/or

c) at least 60% sequence identity with the parent CDR sequence;

preferably wherein the functionally active CDR variant comprises 1 or 2 point mutations in any CDR sequence consisting of less than 4 or 5 amino acids.

Specifically, the functionally active variant antibody comprises at least one of the functionally active CDR variants as described herein. Specifically, the functionally active variant antibody comprising one or more of the functionally active CDR variants has a specificity to bind the same epitope as the parent antibody.

According to a specific aspect, a point mutation is any of an amino acid substitution, deletion and/or insertion of one or more amino acids.

Specifically, the antibody is derived from any of such parent antibodies by mutagenesis, employing the respective CDR sequences, or CDR mutants, including functionally active CDR variants, e.g. with 1, 2 or 3 point mutations within one CDR loop, e.g. within a CDR length of 5-18 amino acids, e.g. within a CDR region of 5-15 amino acids or 5-10 amino acids. Alternatively, there may be 1 to 2 point mutations within one CDR loop, e.g. within a CDR length of less than 5 amino acids, to provide for an antibody comprising a functionally active CDR variant. Specific CDR sequences might be short, e.g. the CDR2 or CDR5 sequences. According to a specific embodiment, the functionally active CDR variant comprises 1 or 2 point mutations in any CDR sequence consisting of less than 4 or 5 amino acids.

It is herein specifically understood that the CDRs numbered CDR1, 2, and 3 represent the binding region of the VH domain, and CDR4, 5, and 6 represent the binding region of the VL domain.

Further specific antibodies are provided as CDR mutated antibodies, e.g. to improve the affinity of an antibody and/or to target the same epitope or epitopes near the epitope that is targeted by a parent antibody (epitope shift), however, still specifically recognizing the gal-II epitope.

Specifically, the VH or heavy chain (HC) sequences of such variants may be substituted by VH and HC sequences of another variant, respectively, in particular where the other variant is any other variant of the same parent antibody.

Specifically, the VL or light chain (LC) sequences of such variants may be substituted by VL and LC sequences of another variant, respectively, in particular where the other variant is any other variant of the same parent antibody.

According to a specific aspect, the antibody comprises recombinant CDR and framework sequences, e.g. of different origin, wherein at least one of the CDR and framework sequences includes human, humanized, chimeric, murine or affinity matured sequences, yet wherein the framework and particularly the Fc region is of an human IgG1 or IgG3, or an Fc region of a human IgG1 or IgG3 constant region variant (allotypes), or of a recombinant IgG1 or IgG3 antibody which comprises a randomized or artificial amino acid sequence (e.g. not naturally-occurring) sequence, however, not changing the IgG1 or IgG3 subtype structure.

Specifically preferred antibodies comprise the binding site of any of the parent antibodies, in particular the binding site formed by the combination of the respective VH and VL domains.

Specifically, the antibody is an engineered mAb comprising one or more (several) point mutations to improve the C1q binding (and optionally the CDC activity) of the antibody, i.e. by engineering the C1q binding site of the Fc region through one or more (several) point mutations or glycostructure. Specifically, the antibody may be engineered to improve CDC activity by improved C1q activation.

The invention further provides for a method of producing functionally active antibody variants of a parent antibody which is any of the 8E9, G2-27, or G2-33 antibodies, or comprising the binding site any of the 8E9, G2-27, or G2-33 antibodies, which method comprises engineering at least one point mutation in any of the constant regions or complementarity determining regions (CDR1 to CDR6) to obtain a variant antibody, and determining the functional activity of the variant antibody, specifically by the affinity to bind the O1 epitope with a Kd of less than 10⁻⁶M, preferably less than 10⁻⁷M, or less than 10⁻⁸M, or less than 10⁻⁹M, even less than 10⁻¹⁰M, or less than 10⁻¹¹ M, e.g. with an affinity in the picomolar range, and by the CDC activity. Upon determining the functional activity, the functionally active variants are selected for further use and optionally for production by a recombinant production method. The variant antibody derived from the parent antibody by mutagenesis may be produced a methods well-known in the art.

According to a specific aspect, the variant antibody binds the same epitope as the parent antibody.

According to a further specific aspect, the variant antibody comprises the same binding site as the parent antibody.

Specifically, the antibody has an affinity to bind the O1 antigen with a Kd of less than 10⁻⁶M, preferably less than 10⁻⁷M or less than 10⁻⁸M.

The antibody as described herein is specifically further characterized that it does not cross-react with any other K. pneumoniae antigen, and/or the antibody binds to any other K. pneumoniae antigen with a lower affinity, e.g. where the Kd difference to preferentially bind the O1 antigen over other K. pneumoniae antigens (other than the O1 antigen) is at least 2 logs, preferably at least 3 logs.

Variants of parent antibodies which are produced by affinity maturation, herein referred to as affinity-maturated variants, may have an increased binding affinity, with a Kd difference of at least 1 log, or 2 logs, or 3 logs, as compared to the parent antibody. Affinity maturated variants typically have an affinity to bind the O1 antigen with a Kd of less than 10⁻⁸M, or less than 10⁻⁹M. If the parent antibody has an affinity with a Kd of less than 10⁻⁸M, or less than 10⁻⁹M, and the parent antibody is undergoing affinity maturation, the affinity matured variant may have an even higher affinity with a Kd of less than 10⁻⁹M and less than 10⁻¹⁰M, respectively.

Specifically, the antibody is a full-length monoclonal antibody, an antibody fragment thereof comprising at least one antibody domain construct incorporating the antigen-binding site and the Fc region, or a fusion protein comprising at least said antibody fragment fused to a heterologous peptide or polypeptide.

Specifically, the antibody comprises the Fc of a human IgG, such as IgG1 or IgG3 preferably any human IgG1 or IgG3 allotype, preferably the Fc region or Fc part of human IgG1, such as an antibody comprising the constant region of human IgG1 allotype G1m1,17 identified by the amino acid sequence SEQ ID 7, or the Fc part or Fc region thereof, e.g. the human IgG1 Fc identified by SEQ ID 8, or a functional variant thereof, comprising a C1q binding site. SEQ ID 7 identifies the G1 m1,17 allotype of human IgG1, SEQ ID 8 identifies the Fc part incorporated within SEQ ID 7.

Alternatively, the Fc or Fc region of any other human IgG1 or IgG3, or Fc variants or constant region variants of any of human IgG1 or IgG3, or allotype of human IgG1 or IgG3 can be used, as long as it comprises a C1q binding site.

The invention further provides for an isolated nucleic acid encoding the antibody as described herein.

The invention further provides for an expression cassette or a plasmid comprising a coding sequence to express the antibody as described herein, or a protein comprising a VH and/or VL of said antibody and the Fc region.

The invention further provides for a host cell comprising the nucleic acid or the an expression cassette or a plasmid as described herein.

The invention further provides for a method of producing the antibody as described herein, wherein a host cell as described herein is cultivated or maintained under conditions to produce said antibody. Thus, the invention provides for a method of producing the antibody as described herein, wherein a recombinant host cell capable of expressing the antibody is cultivated or maintained under conditions to produce said antibody.

Specifically preferred is a host cell and a production method employing such host cell, which host cell comprises
the plasmid or expression cassette as described herein, which incorporates a coding sequence to express the antibody light chain; and
the plasmid or expression cassette as described herein, which incorporates a coding sequence to express the antibody heavy chain.

According to a further aspect, the invention provides for a method of producing an antibody as described herein, comprising

a) immunizing a non-human animal with the O1 antigen of Klebsiella pneumoniae and isolating B-cells producing antibodies;

b) forming immortalized cell lines from the isolated B-cells;

c) screening the cell lines to identify a cell line producing a monoclonal antibody that specifically binds to the O1 antigen; and

d) producing a humanized or human IgG1 or IgG3 form of the antibody, or an IgG1 or IgG3 derivative thereof with the same epitope binding specificity as the monoclonal antibody.

Specific methods include a process for producing switch variant clones producing class IgG, such as substantially encoded by the immunoglobulin gamma gene, and subclass IgG1 or IgG3.

The invention further provides for a method of identifying a candidate antibody comprising:

a) providing a sample containing an antibody or antibody-producing cell; and

b) assessing for

i. binding of an antibody in or produced by the sample with a galactan-II epitope; and

ii. CDC activity for killing of K. pneumoniae O1 serotype in a serum sample;

wherein a positive binding reaction between the antibody and the epitope, and the positive CDC activity identifies the antibody as candidate antibody.

The invention further provides for a method of producing an antibody as described herein, comprising

a) providing a candidate antibody identified as described herein; and

b) producing a humanized or human IgG1 or IgG3 form of the antibody, or an IgG1 or IgG3 derivative thereof with the same epitope binding specificity as the monoclonal antibody.

The invention further provides for an artificial composition comprising the monoclonal antibody described herein, in particular an antibody produced by a recombinant host cell and isolated from a host cell culture. Such composition specifically does not comprise any human serum protein, which would contaminate the composition. In particular, the composition is a monoclonal antibody composition comprising a single set of monoclonal antibodies only. Therefore, the composition is considered artificial and not naturally-occurring.

The invention further provides for a pharmaceutical preparation comprising the antibody as described herein, preferably comprising a parenteral or mucosal formulation, optionally containing a pharmaceutically acceptable carrier or excipient.

Such pharmaceutical composition may contain the antibody as the sole active substance, or in combination with other active substances, or a cocktail of active substances, such as a combination or cocktail of at least two or three different antibodies.

According to the invention, the antibody of the invention is specifically provided for medical, diagnostic or analytical use.

The invention further provides for the medical use of the antibody described herein, and the respective method of treating a subject in need of immunoprophylaxis or therapy.

Specifically, the invention provides for the antibody as described herein, for use in treating a subject at risk of or suffering from Klebsiella pneumoniae infection or colonization comprising administering to the subject an effective amount of the antibody to limit the infection in the subject or to ameliorate a disease condition resulting from said infection, preferably for treatment or prophylaxis of any of primary and secondary bacteremia, pneumonia, urinary tract infection, liver abscess, peritonitis, or meningitis.

Accordingly, the invention provides for a method of treating a subject at risk of or suffering from Klebsiella pneumoniae infection or colonization comprising administering to the subject an effective amount of the antibody to limit the infection in the subject or to ameliorate a disease condition resulting from said infection, preferably for treatment or prophylaxis of any of primary and secondary bacteremia, pneumonia, urinary tract infection, liver abscess, peritonitis, or meningitis.

Specifically, the subject is an immunocompromised or immunosuppressed patient, or a contact thereof.

Specifically, the subject is of a host group characterized by an impaired phagocyte number and/or function, which host group is any of

a) patients suffering from inherited or acquired primary or secondary immunodeficiency;

b) patients selected from the group consisting of neonates younger than x months of age, elderly patients older than 65 years of age, patients suffering from Diabetes mellitus, renal failure, cirrhosis, Trisomie 21, trauma, or HIV, or patients who have undergone surgical interventions or systemic treatment with corticosteroids; or

c) patients admitted to hospital or hospital personnel, in particular at an acute-care or intensive care unit, with a risk of contracting infection upon exposure to a patient suffering from K. pneumoniae disease.

Specifically, the antibody is used to prevent nosocomial or iatrogenic outbreaks of K. pneumoniae disease.

Specifically, the antibody is provided for use according to the invention, wherein a systemic infection or colonization with Klebsiella pneumoniae of the gal-II O-type in a subject is determined ex vivo by contacting a biological sample of said subject with the antibody, wherein a specific immune reaction of the antibody determines the infection or colonization.

Specifically, the biological samples is a body fluid or tissue sample, preferably a sample selected from the group consisting of a blood sample, stool sample, skin sample, urine sample, cerebrospinal fluid, and a respiratory tract specimen such as endotracheal aspirates, pleural fluid, lung tap, nasal swab or sputum, or a Klebsiella pneumoniae isolate originating from any of the foregoing. Specifically, a sample of body fluid is tested for the specific immune reaction, which sample is selected from the group consisting of urine, blood, blood isolates or blood culture, aspirate, sputum, lavage fluid of intubated subjects and stool.

Specifically, the biological sample is treated to produce a Klebsiella pneumoniae isolate originating from the biological sample, which isolate may be further characterized for its gal-II genotype or phenotype, and/or the level of O1 (D-gal-II) antigen expression. Preferable sample preparation methods for producing bacterial isolates are employing bacterial enrichment and cultivation steps.

Specifically, the biological sample is treated to determine the O1 level directly in the sample, optionally following preparatory steps of enrichment or purification to reduce matrix effects and to increase the specificity and sensitivity of the test. Preparatory steps include culturing of the biological specimen according to standard culture procedures such as but not exclusively being hemocultures in standard growth media as well as the culturing of specimens on solid agar (including phenotyping—i.e. antibiogram) as performed in routine microbiology laboratories. Bacteria may be sub-cultured for expansion of CFU in different growth media (standard media and/or chemically defined media; high nutrient, low nutrient, limited growth media composition) to enhance expression of virulence factors. Bacterial suspensions may be prepared and washed in standard buffer solutions to remove potential matrix effects.

Specifically, the O1 antigen is determined by at least one of an immunoassay, preferably any of ELISA, CIA, RIA, IRMA, agglutination assay, immunochromatography, dipstick assay and Western-blot, or mass-spectrometry, nuclear magnetic resonance (NMR), or a method of determining corresponding DNA or RNA indicative of O1 expression, preferably employing a nucleic acid hybridization assay or a nucleic acid amplification assay.

According to a specific aspect, immunotherapy using the antibody of the invention may effectively protect against live bacterial challenge, e.g. as determined in various animal models.

The antibody is specifically effective against Klebsiella pneumoniae of the gal-II O-type by its CDC activity or complement-mediated killing, e.g. as determined by an in vitro serum bactericidal assay (SBA), e.g. with at least 20% killing of bacteria above the control samples (no antibody or irrelevant control mAb added).

The antibody is specifically effective against Klebsiella pneumoniae of the gal-II O-type by antibody mediated phagocytosis, e.g. as determined by an in vitro opsonophagocytotic killing assay (OPK), e.g. with at least 20% uptake of input bacteria or 20% lower end CFU count above the control samples (no antibody or irrelevant control mAb added).

According to a further specific aspect, the antibody is bactericidal in vitro and/or in vivo, and is specifically killing the targeted pathogen in animals, including both, human and non-human animals, and inhibits pathogenesis in vivo, preferably any models of primary and secondary bacteremia, pneumonia, urinary tract infection, liver abscess, peritonitis, or meningitis.

According to a specific embodiment, the antibody is administered at a prophylactically effective dose to prevent bacteremia, preferably less than 1 mg/kg.

According to another specific embodiment, the antibody is administered in a therapeutically effective dose to treat bacteremia, preferably less than 10 mg/kg.

Specifically, the antibody is administered in a pharmaceutical preparation comprising the antibody and a pharmaceutically acceptable carrier.

Specifically, the antibody is administered in combination with an antibiotic drug. Exemplary antibiotics used for combination with the immunotherapy are those typically used for treating patients with K. pneumoniae infection, e.g. any one or more of carbapenems, polymixins, tygecycline, or betalactams with non-beta lactam type inhibitors.

According to the invention, the antibody as described herein is specifically provided for medical, diagnostic or analytical use.

The invention further provides for the use of the antibody as described herein for diagnostic purposes, specifically for the diagnosis of Klebsiella pneumoniae infection or colonization, or an associated disease such as primary and secondary bacteremia, pneumonia, urinary tract infection, liver abscess, peritonitis, or meningitis in a subject.

Specifically, the subject is a human being, in particular an immunocompromised or immunosuppressed patient, or a contact thereof.

Specifically, the antibody is provided for use as described herein, wherein a systemic infection or colonization with Klebsiella pneumoniae of the gal-II O-type in a subject is determined ex vivo by contacting a biological sample of said subject with the antibody, wherein a specific immune reaction of the antibody determines the infection or colonization.

Specifically, the biological samples is a body fluid or tissue sample, preferably a sample selected from the group consisting of a blood sample, stool sample, skin sample, urine sample, cerebrospinal fluid, and a respiratory tract specimen such as endotracheal aspirates, pleural fluid, lung tap, nasal swab or sputum, or a Klebsiella pneumoniae isolate originating from any of the foregoing. Specifically, a sample of body fluid is tested for the specific immune reaction, which sample is selected from the group consisting of urine, blood, blood isolates or blood culture, aspirate, sputum, lavage fluid of intubated subjects and stool.

Specifically, the biological sample is treated to produce a Klebsiella pneumoniae isolate originating from the biological sample, which isolate may be further characterized for its gal-II genotype or phenotype, and/or the level of gal-II antigen expression. Preferable sample preparation methods for producing bacterial isolates are employing bacterial enrichment and cultivation steps.

Specifically, the biological sample is treated to determine the gal-II level directly in the sample, optionally following preparatory steps of enrichment or purification to reduce matrix effects and to increase the specificity and sensitivity of the test. Preparatory steps include culturing of the biological specimen according to standard culture procedures such as but not exclusively being hemocultures in standard growth media as well as the culturing of specimens on solid agar (including phenotyping—i.e. antibiogram) as performed in routine microbiology laboratories. Bacteria may be sub-cultured for expansion of CFU in different growth media (standard media and/or chemically defined media; high nutrient, low nutrient, limited growth media composition) to enhance expression of virulence factors. Bacterial suspensions may be prepared and washed in standard buffer solutions to remove potential matrix effects.

Specifically, the gal-II antigen is determined by at least one of an immunoassay, preferably any of ELISA, CIA, RIA, IRMA, agglutination assay, immunochromatography, dipstick assay and Western-blot, or mass-spectrometry, nuclear magnetic resonance (NMR), or a method of determining corresponding DNA or RNA indicative of gal-II expression, preferably employing a nucleic acid hybridization assay or a nucleic acid amplification assay.

Specifically, the diagnostic use according to the invention refers to determining the serotype of Klebsiella pneumoniae in vitro from a pure Klebsiella pneumoniae culture recovered from a clinical specimen, to determine whether the bacterium is of the O1 type (i.e. expresses gal-II), or not.

The invention further provides for a diagnostic preparation of the antibody as described herein, comprising the antibody and a further diagnostic reagent in a composition or a kit of parts, comprising the components

a) the antibody; and

b) the further diagnostic reagent;

c) and optionally a solid phase to immobilize at least one of the antibody and the diagnostic reagent.

The diagnostic preparation optionally comprises the antibody of the invention and the further diagnostic reagent in a composition or a kit of parts.

The diagnostic kit preferably comprises all essential components to determine the gal-II expression in the biological sample, optionally without common or unspecific substances or components, such as water, buffer or excipients. The storage stable kit can be stored preferably at least 6 months, more preferably at least 1 or 2 years. It may be composed of dry (e.g. lyophilized) components, and/or include preservatives.

The preferred diagnostic kit is provided as a packaged or prepackaged unit, e.g. wherein the components are contained in only one package, which facilitates routine experiments. Such package may include the reagents necessary for one or more tests, e.g. suitable to perform the tests of a series of biological samples. The kit may further suitably contain a gal-II antigen preparation as a standard or reference control.

The diagnostic composition may be a reagent ready-to-use in a reaction mixture with the biological sample, or a conserved form of such reagent, e.g. a storage-stable form such as lyophilized; snap-frozen (e.g. in liquid nitrogen), ultra low-temperature storage (−70° C. and −80° C.), cold-storage (−20° C. and 5° C.) and controlled room temperature (15° C.-27° C.); standard sample storage as e.g. glycerol-stocks, tissue paraffin-blocks, (buccal) swabs and other standard biological sample storage methods, which conserved form of a reagent can be reconstituted or prepared to obtain a ready-to-use reagent. Such ready-to-use reagent is typically in the form of an aqueous solution, specifically (physiological) buffer conditions (e.g. EDTA buffered, phosphate buffer, HBSS, citrate buffer etc.).

Specifically, the further diagnostic reagent is a reagent specifically reacting with the antibody and/or the reaction product of the antibody binding to its antigen. An appropriate diagnostic reagent is suitably used for performing an immunoassay for diagnosing or monitoring, in a subject, the Klebsiella pneumoniae O1 infection or colonization. The appropriate diagnostic reagent can be a solvent, a buffer, a dye, an anticoagulant, a ligand that specifically binds to the antibody of the invention and/or the antibody-antigen immune complex.

Specifically, the invention provides for a diagnostic preparation of an antibody of the invention, optionally containing the antibody with a label and/or a further diagnostic reagent with a label, such as a reagent specifically recognizing the antibody or an immune complex of the antibody with the respective target antigen, and/or a solid phase to immobilize at least one of the antibody and the diagnostic reagent.

The antibody or the diagnostic reagent can be directly labeled or indirectly labeled. The indirect label may comprise a labeled binding agent that forms a complex with the antibody or diagnostic reagent to the gal-II antigen.

The label is typically a molecule or part of a molecule that can be detected in an assay. Exemplary labels are chromophores, fluorochromes, or radioactive molecules. In some embodiments the antibody or diagnostic reagent is conjugated to a detectable label which may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Preferred diagnostic preparations or assays comprise the antibody of the invention immobilized on a solid phase, e.g. latex beads, gold particles, etc., e.g. to test agglutination by the antibody of bacteria of the gal-II type obtained from a sample to be tested.

The invention further provides for a method of diagnosing Klebsiella pneumoniae O1 (O1 serotype) infection or colonization in a subject caused by a Klebsiella pneumoniae O1 strain, comprising

a) providing an antibody according to the invention, and

b) detecting if the antibody specifically immunoreacts with the galactan-II epitope in a biological sample of the subject to be tested, thereby diagnosing Klebsiella pneumoniae O1 infection or colonization.

According to a specific aspect, the invention provides for companion diagnostics to determine the infection of a subject with Klebsiella pneumoniae O1, in particular with MDR Klebsiella pneumoniae, by the diagnostics of the invention or the diagnostic method of the invention, to provide for the basis of treatment with a therapeutic against such infection, e.g. employing immunotherapy, such as treating with an antibody of the invention.

According to a specific aspect, the invention provides for a sensitive bedside diagnostics to diagnose infection of a subject with Klebsiella pneumoniae O1, in particular with MDR Klebsiella pneumoniae, by determining free LPS, e.g. from clinical specimen where the amount of live bacteria is limited. The sensitivity of such assay is specifically less than 100 ng preferably less than 10 ng of LPS.

### FIGURES

FIG. 1. Serotype distribution of MDR Klebsiella isolates from various sources.

FIG. 2. Immunoblot with O1 specific mAb. One μg LPS purified from O1, O2, and O3 strains was separated and blotted onto PVDF membranes. Binding of murine mAb 8E9 to the separated LPS samples was detected with HRP conjugated anti-mouse IgG specific antibodies.

FIG. 3. Surface staining of K. pneumoniae O1 strains with human/murine chimeric (8E9) and humanized (G2-27 and G2-33) O1 specific mAbs. Murine mAb 8E9 was first chimerized (also called 8E9) and subsequently further humanized giving rise to G2-27 and G2-33. These share the same CDR-s with 8E9, however murine framework (FR) sequences were replaced by human sequences.

Surface binding of O1 specific mAbs on strains A) ATCC 43816 (O1:K2) or B) Kp24 (O1:K+) was detected with flow cytometry. Histograms show fluorescence intensity measured.

FIG. 4. Protection by O1 specific mAbs in the mouse model of K. pneumoniae bacteremia. Mice were immunized prophylactically with either chimeric or humanized O1-specific mAbs or with an irrelevant control mAb. Survival was monitored following a subsequent intravenous challenge. Graphs show combined data of three independent experiments with groups of 5 mice each.

FIG. 5. Serum bactericidal assay to determine CDC activity. K. pneumoniae strain PCM-37 (O1:K37) was cultured in two different (panels A: normal human serum #1; and B: normal human serum #2) 50% depleted human serum samples in the presence of various concentrations of specific or control mAbs. Bactericidal activity is expressed as percentage of recovered bacteria at the end vs the beginning (TO) of the incubation period (3 h) as determined by plating aliquots at both timepoints.

FIG. 6. Sequences

SEQ ID 1: VH CDR1=CDR1

SEQ ID 2: VH CDR2=CDR2

SEQ ID 3: VH CDR3=CDR3

SEQ ID 4: VL CDR1=CDR4

SEQ ID 5: VL CDR2=CDR5

SEQ ID 6: VL CDR3=CDR6

SEQ ID 7: Constant region of the human IgG1 (allotype G1m1,17)

SEQ ID 8: Human IgG1 Fc

### DETAILED DESCRIPTION OF THE INVENTION

The term “antibody” as used herein shall refer to polypeptides or proteins that consist of or comprise antibody domains, which are understood as constant and/or variable domains of the heavy and/or light chains of immunoglobulins, with or without a linker sequence. Polypeptides are understood as antibody domains, if comprising a beta-barrel structure consisting of at least two beta-strands of an antibody domain structure connected by a loop sequence. Antibody domains may be of native structure or modified by mutagenesis or derivatization, e.g. to modify the antigen binding properties or any other property, such as stability or functional properties, such as binding to the Fc receptors FcRn and/or Fcgamma receptor.

The antibody as described herein has a specific binding site to bind one or more antigens or one or more epitopes of such antigens, specifically comprising a CDR binding site of pairs of variable antibody domains, i.e. a VL/VH pair, and constant antibody domains, in particular a full length antibody.

The term “antibody” as described herein shall particularly refer to the IgG1 or IgG3 structure, which is herein understood as a structure comprising two VH/VL pairs, wherein each VH domain is part of a HC comprising the VH-CH1-CH2-CH3 domain sequence, and each VL domain is part of a LC comprising the VL-CL (in particular, kappa) domain sequence, with a linking sequence or hinge region, and wherein the CH2-CH3 domains dimerize to form an Fc. Antibodies of the IgG1 or IgG3 structure typically comprises the variable and constant antibody domains with human IgG1 or IgG3 framework sequences, or terminally elongated or shortened sequences of any of the antibody domains. Likewise, loop sequences or beta-barrel sequences may be mutated without impairing the antibody domain tertiary structure. The term “full length antibody” is generally used to refer to any antibody molecule comprising at least the major part of the Fc domain (comprising at least the CH2-CH3 interface region which forms the Fcgamma receptor binding site, and at least the N-terminal CH2 regions which forms the FcRn binding site). The phrase “full length antibody” is specifically used herein to emphasize that a particular antibody molecule is not an antibody fragment devoid of the Fc region, such as a Fab or scFv fragment.

The term “antibody” shall specifically include antibodies in the isolated form, which are substantially free of other antibodies directed against different target antigens or comprising a different structural arrangement of antibody domains. Still, an isolated antibody may be comprised in a combination preparation, containing a combination of the isolated antibody, e.g. with at least one other antibody, such as monoclonal antibodies or antibody fragments having different specificities.

The term “antibody” shall apply to antibodies of animal origin, including human species, such as mammalian, including human, murine, rabbit, goat, lama, cow and horse, or avian, such as hen, which term shall particularly include recombinant antibodies which are based on a sequence of animal origin, e.g. human sequences.

The antibody as described herein specifically is human or humanized. A humanized antibody specifically can be a human/murine or human/non-human chimeric antibody comprising sequences of origin of different species. For example, the human/murine chimeric antibody comprises sequences of both, human and murine origin, and typically comprises a human Fc region, a human Fc or a human constant region including the Fc region or Fc part of the antibody.

The term “human” as used with respect to an antibody, is understood to include antibodies having variable and constant regions derived from human germline immunoglobulin sequences. The human antibody of the invention may include amino acid residues not encoded by human germline immunoglobulin sequences (e.g., mutations introduced by random or site-specific mutagenesis in vitro or by somatic mutation in vivo), for example in the CDRs. Human antibodies include antibodies isolated from human immunoglobulin libraries or from animals transgenic for one or more human immunoglobulin.

The term “humanized” as used with respect to an antibody refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified, e.g. by one or more amino acid substitutions, preferably modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

The term “chimeric” as used with respect to an antibody refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. For example, the variable region can be derived from presently known sources using readily available B-cells or hybridomas from non-human host organisms in combination with constant regions derived from, for example, human cell preparations.

Exemplary human or humanized antibodies can be produced by and isolated from a recombinant host cell transformed to express the antibody, or antibodies isolated from a recombinant, combinatorial library of antibodies or antibody domains, or antibodies prepared, expressed, created or isolated by any other means that involve splicing of antibody gene sequences to other DNA sequences.

It is understood that the term “antibody” also refers to derivatives of an antibody, in particular functionally active derivatives. An antibody derivative is understood as any combination of one or more antibody domains or antibodies and/or a fusion protein, in which any domain of the antibody may be fused at any position of one or more other proteins, such as other antibodies, e.g. a binding structure comprising CDR loops, a receptor polypeptide, but also ligands, scaffold proteins, enzymes, toxins and the like. A derivative of the antibody may be obtained by association or binding to other substances by various chemical techniques such as covalent coupling, electrostatic interaction, di-sulphide bonding etc. The other substances bound to the antibody may be lipids, carbohydrates, nucleic acids, organic and inorganic molecules or any combination thereof (e.g. PEG, prodrugs or drugs). In a specific embodiment, the antibody is a derivative comprising an additional tag allowing specific interaction with a biologically acceptable compound. There is not a specific limitation with respect to the tag usable in the present invention, as far as it has no or tolerable negative impact on the binding of the antibody to its target. Examples of suitable tags include His-tag, Myc-tag, FLAG-tag, Strep-tag, Calmodulin-tag, GST-tag, MBP-tag, and S-tag. In another specific embodiment, the antibody is a derivative comprising a label. The term “label” as used herein refers to a detectable compound or composition which is conjugated directly or indirectly to the antibody so as to generate a “labeled” antibody. The label may be detectable by itself, e.g. radioisotope labels or fluorescent labels, or, in the case of an enzymatic label, may catalyze chemical alteration of a substrate compound or composition which is detectable.

The preferred derivatives as described herein are functionally active with regard to the antigen binding, preferably which have a potency to combat K. pneumoniae, e.g. as determined in a CDC (SBA), and/or OPK assay, or to protect against bacterial challenge.

Specifically, an antibody derived from an antibody of the invention may comprise at least one or more of the CDR regions or CDR variants thereof being functionally active in differentially binding to the O1 antigen, e.g. specifically or selectively binding the O1 antigen.

Antibodies derived from a parent antibody or antibody sequence, such as a parent CDR or FR sequence, are herein particularly understood as mutants or variants obtained by e.g. in silico or recombinant engineering or else by chemical derivatization or synthesis.

It is understood that the term “antibody” also refers to variants of an antibody, including antibodies with functionally active CDR variants of a parent CDR sequence, and functionally active variant antibodies of a parent antibody.

The term “variant” shall particularly refer to antibodies, such as mutant antibodies or fragments of antibodies, e.g. obtained by mutagenesis methods, in particular to delete, exchange, introduce inserts into a specific antibody amino acid sequence or region or chemically derivatize an amino acid sequence, e.g. in the constant domains to engineer the antibody stability, effector function or half-life, or in the variable domains to improve antigen-binding properties, e.g. by affinity maturation techniques available in the art. Any of the known mutagenesis methods may be employed, including point mutations at desired positions, e.g. obtained by randomization techniques. In some cases positions are chosen randomly, e.g. with either any of the possible amino acids or a selection of preferred amino acids to randomize the antibody sequences. The term “mutagenesis” refers to any art recognized technique for altering a polynucleotide or polypeptide sequence. Preferred types of mutagenesis include error prone PCR mutagenesis, saturation mutagenesis, or other site directed mutagenesis.

The term “variant” shall specifically encompass functionally active variants.

The term “functionally active variant” of a CDR sequence as used herein, is understood as a “functionally active CDR variant”, and the “functionally active variant” of an antibody as used herein, is understood as “functionally active antibody variant”. The functionally active variant means a sequence resulting from modification of this sequence (a parent antibody or a parent sequence) by insertion, deletion or substitution of one or more amino acids, or chemical derivatization of one or more amino acid residues in the amino acid sequence, or nucleotides within the nucleotide sequence, or at either or both of the distal ends of the sequence, e.g. in a CDR sequence the N-terminal and/or C-terminal 1, 2, 3, or 4 amino acids, and/or the centric 1, 2, 3, or 4 amino acids (i.e. in the midst of the CDR sequence), and which modification does not affect, in particular impair, the activity of this sequence. In the case of a binding site having specificity to a selected target antigen, the functionally active variant of an antibody would still have the predetermined binding specificity, though this could be changed, e.g. to change the fine specificity to a specific epitope, the affinity, the avidity, the Kon or Koff rate, etc. For example, an affinity matured antibody is specifically understood as a functionally active variant antibody. Hence, the modified CDR sequence in an affinity matured antibody is understood as a functionally active CDR variant.

Specifically, the functionally active variants of an antibody of the invention have the potency to specifically bind gal-II antigen of K. pneumoniae O1, and the CDC activity to kill K. pneumoniae bacteria of the O1 serotype in the circulation/in serum.

Functionally active variants may be obtained, e.g. by changing the sequence of a parent antibody, e.g. an antibody comprising the same binding site as any of the parent antibodies as described herein, but with modifications within an antibody region besides the binding site, or derived from such parent antibody by a modification within the binding site but that does not impair the antigen binding, and preferably would have substantially the same biological activity as the parent antibody or even an improved activity, including the ability to specifically or selectively bind O1 antigen of K. pneumoniae, and the bactericidal CDC activity or potency of complement mediated killing in an SBA assay. Optionally, the functionally active variants may further include a potency of an antibody mediated phagocytosis in an OPK assay, e.g. with substantially the same biological activity, as determined by the specific binding assay or functional test to target (MDR) K. pneumoniae.

Antibodies combating or killing K. pneumoniae are able to limit or prevent infection and/or to ameliorate a disease condition resulting from such infection, or to inhibit K. pneumoniae pathogenesis, in particular dissemination and replication into or within sterile body compartments/sites of the host. In this regard, the bactericidal antibody as described herein which is characterized by the bactericidal CDC activity, is also understood as being a “protective antibody” meaning that the antibody is responsible for immunity to an infectious agent observed in active or passive immunity. In particular, protective antibodies as described herein are possibly used for therapeutic purposes, e.g. for prophylaxis or therapy, to prevent, ameliorate, treat or at least partially arrest disease symptoms, side effects or progression of disease induced by a pathogen. Specifically, protective antibodies are able to kill or impede replication of live K. pneumoniae cells by e.g. inducing CDC or opsonophagocytic activities, or remove whole bacterial cells or the LPS molecules thereof from the sterile body sites following therapeutic applications (i.e. given on an established infection). Alternatively, prophylactically applied protective antibodies inhibit establishment of an infection (i.e. spread of K. pneumoniae from non-sterile sites to sterile body compartments) by one of the abovementioned or other mechanisms.

The term “substantially the same biological activity” as used herein refers to the activity as indicated by substantially the same activity being at least 20%, at least 50%, at least 75%, at least 90%, e.g. at least 100%, or at least 125%, or at least 150%, or at least 175%, or e.g. up to 200%, or even a higher activity as determined for the comparable or parent antibody.

The preferred variants or derivatives as described herein are functionally active with regard to the antigen binding, preferably which have a potency to specifically bind O1antigen, and not binding to other antigens of K. pneumoniae, with a Kd value difference of at least 2 logs, preferably at least 3 logs, and further including a potency of complement mediated killing in an CDC or SBA assay, e.g. to achieve significant reduction in bacterial counts relative to control samples not containing the antibody, and/or optionally further including a potency of an antibody mediated phagocytosis in an OPK assay, such as to achieve significant reduction in bacterial counts relative to control samples not containing the antibody, e.g. with substantially the same biological activity, as determined by the specific binding assay or functional test to target K. pneumoniae. The significant reduction of activity in the various assays typically means the reduction of at least 50%, preferably at least 60%, 70%, 80%, 90%, 95% or 98% up to complete reduction of about 100% (+/−1%).

In a preferred embodiment the functionally active variant of a parent antibody

a) is a biologically active fragment of the antibody, the fragment comprising at least 80% of the sequence of the molecule, preferably at least 90%, or at least 95% and most preferably at least 97%, 98% or 99%;

b) is derived from the antibody by at least one amino acid substitution, addition and/or deletion, wherein the functionally active variant has a sequence identity to the molecule or part of it, such as an antibody of at least 50% sequence identity, preferably at least 60%, more preferably at least 70%, more preferably at least 80%, still more preferably at least 90%, even more preferably at least 95% and most preferably at least 97%, 98% or 99%; and/or

c) consists of the antibody or a functionally active variant thereof and additionally at least one amino acid or nucleotide heterologous to the polypeptide or the nucleotide sequence.

In one preferred embodiment of the invention, the functionally active variant of the antibody as described herein is essentially identical to the variant described above, but differs from its polypeptide or the nucleotide sequence, respectively, in that it is derived from a homologous sequence of a different species. These are referred to as naturally occurring variants or analogs.

The term “functionally active variant” also includes naturally occurring allelic variants, as well as mutants or any other non-naturally occurring variants. As is known in the art, an allelic variant is an alternate form of a (poly) peptide that is characterized as having a substitution, deletion, or addition of one or more amino acids that does essentially not alter the biological function of the polypeptide.

Functionally active variants may be obtained by sequence alterations in the polypeptide or the nucleotide sequence, e.g. by one or more point mutations, wherein the sequence alterations retains or improves a function of the unaltered polypeptide or the nucleotide sequence, when used in combination of the invention. Such sequence alterations can include, but are not limited to, (conservative) substitutions, additions, deletions, mutations and insertions.

Specific functionally active variants are CDR variants. A CDR variant includes an amino acid sequence modified by at least one amino acid in the CDR region, wherein said modification can be a chemical or a partial alteration of the amino acid sequence, which modification permits the variant to retain the biological characteristics of the unmodified sequence. A partial alteration of the CDR amino acid sequence may be by deletion or substitution of one to several amino acids, e.g. 1, 2, 3, 4 or 5 amino acids, or by addition or insertion of one to several amino acids, e.g. 1, 2, 3, 4 or 5 amino acids, or by a chemical derivatization of one to several amino acids, e.g. 1, 2, 3, 4 or 5 amino acids, or combination thereof. The substitutions in amino acid residues may be conservative substitutions, for example, substituting one hydrophobic amino acid for an alternative hydrophobic amino acid.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a polynucleotide that results in the expression of an amino acid sequence that differs from the non-engineered amino acid sequence in the substitution or exchange, deletion or insertion of one or more single (non-consecutive) or doublets of amino acids for different amino acids.

Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

The “neutral” amino acids are shown below along with their respective three-letter and single-letter code and polarity:

Alanine: (Ala, A) nonpolar, neutral;

Asparagine: (Asn, N) polar, neutral;

Cysteine: (Cys, C) nonpolar, neutral;

Glutamine: (Gln, Q) polar, neutral;

Glycine: (Gly, G) nonpolar, neutral;

Isoleucine: (Ile, I) nonpolar, neutral;

Leucine: (Leu, L) nonpolar, neutral;

Methionine: (Met, M) nonpolar, neutral;

Phenylalanine: (Phe, F) nonpolar, neutral;

Proline: (Pro, P) nonpolar, neutral;

Serine: (Ser, S) polar, neutral;

Threonine: (Thr, T) polar, neutral;

Tryptophan: (Trp, W) nonpolar, neutral;

Tyrosine: (Tyr, Y) polar, neutral;

Valine: (Val, V) nonpolar, neutral; and

Histidine: (His, H) polar, positive (10%) neutral (90%).

The “positively” charged amino acids are:

Arginine: (Arg, R) polar, positive; and

Lysine: (Lys, K) polar, positive.

The “negatively” charged amino acids are:

Aspartic acid: (Asp, D) polar, negative; and

Glutamic acid: (Glu, E) polar, negative.

“Percent (%) amino acid sequence identity” with respect to the antibody sequences and homologs described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

An antibody variant is specifically understood to include homologs, analogs, fragments, modifications or variants with a specific glycosylation pattern, e.g. produced by glycoengineering, which are functional and may serve as functional equivalents, e.g. binding to the specific targets and with functional properties.

Specific antibodies may be engineered to incorporate modifications to increase Fc effector functions, in particular to enhance CDC activity and/or OPK activity.

Such modifications may be effected by mutagenesis, e.g. mutations in the Fcgamma receptor binding site or by derivatives or agents to interfere with CDC activity of an antibody format, so to achieve increase of Fc effector function.

A significant increase of Fc effector function is typically understood to refer to an increase in Fc effector function of at least 10% of the unmodified (wild-type) format, preferably at least 20%, 30%, 40% or 50%, as measured by CDC or OPK activity.

The term “glycoengineered” variants with respect to antibody sequences shall refer to glycosylation variants having modified immunogenic or immunomodulatory (e.g. anti-inflammatory) properties, CDC, as a result of the glycoengineering. All antibodies contain carbohydrate structures at conserved positions in the heavy chain constant regions, with each isotype possessing a distinct array of N-linked carbohydrate structures, which variably affect protein assembly, secretion or functional activity. IgG1 or IgG3 type antibodies are typically glycoproteins that have a conserved N linked glycosylation site at Asn297 in their CH2 domain. The two complex bi-antennary oligosaccharides attached to Asn297 are buried between the CH2 domains, forming extensive contacts with the polypeptide backbone, and their presence is essential for the antibody to mediate effector functions such as CDC or OPK. Removal of N-Glycan at N297, e.g. through mutating N297, e.g. to A, or T299 typically results in aglycosylated antibody formats with reduced CDC and OPK. Specifically, the antibody of the invention may be glycosylated or glycoengineered.

Major differences in antibody glycosylation occur between cell lines, and even minor differences are seen for a given cell line grown under different culture conditions. Expression in bacterial cells typically provides for an aglycosylated antibody. CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180). In addition to the choice of host cells, factors that affect glycosylation during recombinant production of antibodies include growth mode, media formulation, culture density, oxygenation, pH, purification schemes and the like.

The term “antigen-binding site” or “binding site” refers to the part of an antibody that participates in antigen binding. The antigen binding site is formed by amino acid residues of the N-terminal variable (“V”) regions of the heavy (“H”) and/or light (“L”) chains, or the variable domains thereof. Three highly divergent stretches within the V regions of the heavy and light chains, referred to as “hypervariable regions”, are inter-posed between more conserved flanking stretches known as framework regions, The antigen-binding site provides for a surface that is complementary to the three-dimensional surface of a bound epitope or antigen, and the hypervariable regions are referred to as “complementarity-determining regions”, or “CDRs.” The binding site incorporated in the CDRs is herein also called “CDR binding site”.

The term “antigen” as used herein interchangeably with the terms “target” or “target antigen” shall refer to a whole target molecule or a fragment of such molecule recognized by an antibody binding site. Specifically, substructures of an antigen, e.g. a polypeptide or carbohydrate structure, generally referred to as “epitopes”, e.g. B-cell epitopes or T-cell epitope, which are immunologically relevant, may be recognized by such binding site. Specific antigens like the gal-II antigens are carbohydrate structures and may be provided as isolated antigens optionally provided on an artificial carrier, or else in the form of K. pneumoniae cells expressing the antigens or cell fractions thereof.

The term “epitope” as used herein shall in particular refer to a molecular structure which may completely make up a specific binding partner or be part of a specific binding partner to a binding site of an antibody. An epitope may either be composed of a carbohydrate, a peptidic structure, a fatty acid, an organic, biochemical or inorganic substance or derivatives thereof and any combinations thereof. Epitopes can be either linear or conformational epitopes. A linear epitope is comprised of a single segment of a primary sequence of a polypeptide or carbohydrate chain. Linear epitopes can be contiguous or overlapping.

Conformational epitopes are comprised of amino acids or carbohydrates brought together by folding the polypeptide to form a tertiary structure and the amino acids are not necessarily adjacent to one another in the linear sequence. Specifically and with regard to polypeptide antigens a conformational or discontinuous epitope is characterized by the presence of two or more discrete amino acid residues, separated in the primary sequence, but assembling to a consistent structure on the surface of the molecule when the polypeptide folds into the native protein/antigen.

Herein the term “epitope” shall particularly refer to the single epitope recognized by an antibody.

The term “expression” is understood in the following way. Nucleic acid molecules containing a desired coding sequence of an expression product such as e.g. an antibody as described herein, and control sequences such as e.g. a promoter in operable linkage, may be used for expression purposes. Hosts transformed or transfected with these sequences are capable of producing the encoded proteins. In order to effect transformation, the expression system may be included in a vector; however, the relevant DNA may also be integrated into the host chromosome. Specifically the term refers to a host cell and compatible vector under suitable conditions, e.g. for the expression of a protein coded for by foreign DNA carried by the vector and introduced to the host cell.

Coding DNA is a DNA sequence that encodes a particular amino acid sequence for a particular polypeptide or protein such as e.g. an antibody. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes.

“Vectors” used herein are defined as DNA sequences that are required for the transcription of cloned recombinant nucleotide sequences, i.e. of recombinant genes and the translation of their mRNA in a suitable host organism.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “host cell” as used herein shall refer to primary subject cells transformed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “isolated” or “isolation” as used herein with respect to a nucleic acid, an antibody or other compound shall refer to such compound that has been sufficiently separated from the environment with which it would naturally be associated, so as to exist in “substantially pure” form. “Isolated” does not necessarily mean the exclusion of artificial or synthetic mixtures with other compounds or materials, or the presence of impurities that do not interfere with the fundamental activity, and that may be present, for example, due to incomplete purification. In particular, isolated nucleic acid molecules of the present invention are also meant to include those which are not naturally occurring, e.g. codon-optimized nucleic acids or cDNA, or chemically synthesized.

Likewise, the isolated antibody of the invention is specifically non-naturally occurring, e.g. as provided in a combination preparation with another antibody or active agent, which combination does not occur in nature, or an optimized or affinity-maturated variant of a naturally occurring antibody, or an antibody with a framework-region which is engineered to improve the manufacturability of the antibody. By such optimizing or engineering the antibody comprises one or more synthetic sequences or characteristics, which would not be found in the context of the antibody in nature.

With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

With reference to polypeptides or proteins, such as isolated antibodies or epitopes of the invention, the term “isolated” shall specifically refer to compounds that are free or substantially free of material with which they are naturally associated such as other compounds with which they are found in their natural environment, or the environment in which they are prepared (e g. cell culture) when such preparation is by recombinant DNA technology practiced in vitro or in vivo. Isolated compounds can be formulated with diluents or adjuvants and still for practical purposes be isolated—for example, the polypeptides or polynucleotides can be mixed with pharmaceutically acceptable carriers or excipients when used in diagnosis or therapy. In particular, the isolated antibody of the invention differs from polyclonal serum preparations raised against K. pneumoniae strains, because it is provided in the isolated and purified form, preferably provided in a preparation comprising the isolated antibody as the only active substance. This does not preclude, however, that the isolated antibody is provided in a combination product comprising a limited number of further well-defined (isolated) antibodies. Isolated antibodies may as well be provided on a solid, semi-liquid or liquid carrier, such as beads.

The term “Fc region” as used herein shall refer to the portion of an antibody that correlates to a crystallizable fragment obtained by papain digestion of an IgG molecule. The Fc region consists of the C-terminal region of an IgG heavy chain-made up of the C-terminal approximately half of the two heavy chains of an IgG molecule that are linked by disulfide bonds. The Fc may include the hinge region, or part of the hinge region, which is the proline-rich portion of an immunoglobulin heavy chain between the Fc and Fab regions. The Fc region of an IgG comprises two constant domains, CH2 and CH3. The Fc region has no antigen binding activity and is comprises the carbohydrate moiety and the binding site for the Fc receptor, including the neonatal Fc receptor (FcRn). As an example, the antibody comprising the human IgG1 Fc region contains the wild-type constant region or Fc of human IgG1 SEQ ID 7 and SEQ ID 8, respectively, or a variant thereof comprising the Fc sequence that differs from that of the wild-type Fc sequence by virtue of at least one amino acid modification.

The Fc region of an antibody can interact with a number of Fc receptors, including e.g., FcγRIs, FcγRIIs, FcγRIIs, FcRn, C1q, C3, mannan binding lectin, mannose receptor, staphylococcal protein A, streptococcal protein G, and viral FcγR. For the IgG class the Fc gamma receptors (FcγRs) are important, specifically regarding the complement-mediated bactericidal activity.

Formation of the Fc/FcγR complex recruits effector cells to sites of bound antigen, typically resulting in signaling events within the cells and important subsequent immune responses such as release of inflammation mediators, B cell activation, endocytosis, phagocytosis, and cytotoxic attack.

The different IgG subclasses have different affinities for the FcγRs, with IgG1 and IgG3 typically binding substantially better to the receptors than IgG2 and IgG4.

In complement-dependent cytotoxicity, the C1q binds the antibody and this binding triggers the complement cascade which leads to the formation of the membrane attack complex (MAC) (C5b to C9) at the surface of the target cell, as a result of the classical pathway complement activation. A specific site on Fc serves as the interface for the complement protein C1q. Amino acid residues necessary for C1q binding of human IgG1 and IgG3 are located in the CH2 domain.

Human IgG1 and IgG3 are usually the most efficient of the four human IgG subclasses in activating complement. Typically, the C1q binding site on IgG molecules involves at least one or more of residues Glu 233-Pro238, Phe241, Va1264-Asp270, Tyr296-Asn297, Lys322, Pro329-Glu333 in the CH2 domain. Residues in the hinge region of IgG1 (such as Glu216-Pro232) and IgG3 (such as Glu216-Pro277) may also be important in C1q binding.

A site on the IgG Fc portion (between the CH2 and CH3 domains) mediates the interaction with the neonatal Fc receptor FcRn, the binding of which recycles endocytosed antibody from the endosome back to the bloodstream. The binding site for FcRn on the IgG Fc is also the site at which the bacterial proteins A and G bind.

A specific feature of the Fc region is the conserved N-linked glycosylation that occurs at N297. This carbohydrate plays a structural and functional role for the antibody, and is one of the principle reasons that antibodies are produced using mammalian expression systems. Efficient binding of the IgG Fc domain to FcγR and C1q specifically requires glycosylation. Alterations in the composition of the N297 carbohydrate or its elimination could affect the binding of the Fc to FcγR or C1q.

The term “biological sample” as used herein shall refer to any material obtained from a subject, such as a human being, that contains, or potentially contains, biological material which could contain K. pneumoniae. The biological sample can be a tissue, fluid or cell culture sample. Examples of samples for use in accordance with the invention include, but are not limited to patient samples, e.g., tissue or body fluids, specifically a respiratory tract specimen such as endotracheal aspirates, pleural fluid, lung tap, nasal swab or sputum, a blood sample, stool sample, skin and urine sample or cerebrospinal fluid.

The biological sample typically comprises a complex biological matrix such as complex viscous biological fluids containing multiple types of biological and small organic molecules, for example mucous exudates rich in protein matter. Suitable additives or extraction procedures may be used to reduce the non-specific binding that can be associated with a matrix in the sample and/or lower the matrix viscosity by solubilizing and/or breaking down viscous or solid components of the sample matrix. Sample preparation methods may be employed that liberate markers from organisms and/or break down and/or liquefy biological matrices. Biological matrices that may be analyzed include mucus-containing samples such as nasal secretions, sputum, phlegm, pharyngeal exudates, urethral or vaginal secretions, and washes of such membrane surfaces.

Suitable sample preparation methods include method steps to reduce the effect of the biological matrix on the assay. Such method steps may include but are not limited to, e.g., capture, chromatography, spin-centrifugation and dialysis.

The material obtained from a subject may also be in the form of bacterial isolates, e.g., in the form of a cell culture for cultivating the isolated K. pneumoniae or a cell culture product. Culture media may be selective to enrich solely the K. pneumoniae population, or non-selective.

Bacterial isolate preparation typically involves an incubating step to maintain the sample in conditions that enhance the proliferation of K. pneumoniae, thereby enriching the K. pneumoniae population in the sample.

Once the isolate is obtained, the bacterium may be further investigated by biochemical and/or serological tests, e.g., to determine the O type, and the level of gal-II expressed. Several typing methods are available to study K. pneumoniae strains. These methods typically include serotyping, toxin-typing, standard typing for genetic relationship/phylogeny including multi-locus sequence typing (MLST), or Pulsed Field Gel Electrophoresis (PFGE).

The term O1 antigen, also referred to as “galactan-II”, “gal-II” or “D-gal II” as used herein shall refer to the carbohydrate structure of the LPS O-antigen of K. pneumoniae comprising a galactose polymer and a structure comprising at least one of the repeat unit: [-3)-α-D-Galp-(1-3)-β-D-Galp-(1-].

The respective O-antigen comprising the gal-II structure is herein referred to as “O1 antigen” which includes the “gal-II epitope” being recognized by a O1 specific antibody as described herein. The O1 antigen is understood as the outer part of the LPS of K. pneumoniae of the O1-type (K. pneumoniae O1), which is the surface accessible antigenic carbohydrate structure comprising one or more specific gal-II epitopes incorporated therein.

“Specific” binding, recognizing or targeting as used herein, means that the binder, e.g., antibody or antigen-binding portion thereof, exhibits appreciable affinity for the target antigen or a respective epitope in a heterogeneous population of molecules. Thus, under designated conditions (e.g., immunoassay), a binder specifically binds to the target gal-II antigen and does not bind in a significant amount to other molecules present in a sample. The specific binding means that binding is selective in terms of target identity, high, medium or low binding affinity or avidity, as selected. Selective binding is usually achieved if the binding constant or binding dynamics is at least 10-fold different (understood as at least 1 log difference), preferably the difference is at least 100-fold (understood as at least 2 logs difference), and more preferred a least 1000-fold (understood as at least 3 logs difference) as compared to another target.

Preferred antibodies of the invention are specifically binding the O1 antigen, with a high affinity, in particular with a high on and/or a low off rate, or a high avidity of binding. The binding affinity of an antibody is usually characterized in terms of the concentration of the antibody, at which half of the antigen binding sites are occupied, known as the dissociation constant (Kd, or KD). Usually a binder is considered a high affinity binder with a Kd<10⁻⁷ M, in some cases, e.g. for therapeutic purposes with higher affinities, e.g. with a Kd<10⁻⁸ M, preferably a Kd<10⁻⁹ M, even more preferred is a Kd<10⁻¹⁰ M.

Affinity maturation is the process by which antibodies with increased affinity for a target antigen are produced. Any one or more methods of preparing and/or using affinity maturation libraries available in the art may be employed in order to generate affinity matured antibodies in accordance with various embodiments of the invention disclosed herein. Exemplary such affinity maturation methods and uses, such as random mutagenesis, bacterial mutator strains passaging, site-directed mutagenesis, mutational hotspots targeting, parsimonious mutagenesis, antibody shuffling, light chain shuffling, heavy chain shuffling, CDR1 and/or CDR1 mutagenesis, and methods of producing and using affinity maturation libraries amenable to implementing methods and uses in accordance with various embodiments of the invention disclosed herein, include, for example, those disclosed in: Prassler et al. (2009); Immunotherapy, Vol. 1(4), pp. 571-583; Sheedy et al. (2007), Biotechnol. Adv., Vol. 25(4), pp. 333-352; WO2012/009568; WO2009/036379; WO2010/105256; US2002/0177170; WO2003/074679.

With structural changes of an antibody, including amino acid mutagenesis or as a consequence of somatic mutation in immunoglobulin gene segments, variants of a binding site to an antigen are produced and selected for greater affinities. Affinity matured antibodies may exhibit a several logfold greater affinity than a parent antibody. Single parent antibodies may be subject to affinity maturation. Alternatively pools of antibodies with similar binding affinity to the target antigen may be considered as parent structures that are varied to obtain affinity matured single antibodies or affinity matured pools of such antibodies.

The preferred affinity maturated variant of an antibody according to the invention exhibits at least a 2 fold increase in affinity of binding, preferably at least a 5, preferably at least 10, preferably at least 50, or preferably at least 100 fold increase. The affinity maturation may be employed in the course of the selection campaigns employing respective libraries of parent molecules, either with antibodies having medium binding affinity to obtain the antibody of the invention having the specific target binding property of a binding affinity Kd<10⁻⁸ M. Alternatively, the affinity may be even more increased by affinity maturation of the antibody according to the invention to obtain the high values corresponding to a Kd of less than 10⁻⁹ M, preferably less than 10⁻¹⁰ M or even less than 10⁻¹¹ M, most preferred in the picomolar range. In certain embodiments binding affinity is determined by an affinity ELISA assay.

In certain embodiments binding affinity is determined by a BIAcore, ForteBio or MSD assays. In certain embodiments binding affinity is determined by a kinetic method. In certain embodiments binding affinity is determined by an equilibrium/solution method.

Use of the term “having the same specificity”, “having the same binding site” or “binding the same epitope” indicates that equivalent monoclonal antibodies exhibit the same or essentially the same, i.e. similar immunoreaction (binding) characteristics and compete for binding to a pre-selected target binding sequence. The relative specificity of an antibody molecule for a particular target can be relatively determined by competition assays, e.g. as described in Harlow, et al., ANTIBODIES: A LABORATORY MANUAL, Cold Spring Harbor Laboratory Press, Cold Spring Harbor, N.Y., 1988).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “compete” with each other for binding of their respective epitope(s). Antibodies that compete with any of the exemplified antibodies for binding the gal-II antigen are particularly encompassed by the present invention.

Competition herein means a greater relative inhibition than about 30% as determined by competition ELISA analysis or by ForteBio analysis. It may be desirable to set a higher threshold of relative inhibition as criteria of what is a suitable level of competition in a particular context, e.g., where the competition analysis is used to select or screen for new antibodies designed with the intended function of the binding of the antigen. Thus, for example, it is possible to set criteria for the competitive binding, wherein at least 40% relative inhibition is detected, or at least 50%, at least 60%, at least 70%, at least 80%, at least 90% or even at least 100%, before an antibody is considered sufficiently competitive.

The term “diagnostic kit” as used herein refers to a kit or set of parts, which in combination or mixture can be used to carry out the measurement/detection of one or more analytes or markers to determine a disease or disease condition, or to predict the disease or the disease progression. In particular, the kit contains at least a detection molecule and/or a binder, wherein the detection molecule and/or the binder specifically recognizes the analyte or marker, or a reaction product of such analyte or marker. In addition, various reagents or tools may be included in the kit. The diagnostic kit may comprise any useful reagents for carrying out the subject methods, including substrates such as microbeads or planar arrays or wells, reagents for biomarker isolation, detection molecules directed to specific targets, reagents such as primers for nucleic acid sequencing or amplification, arrays for nucleic acid hybridization, detectable labels, solvents or buffers and the like, various linkers, various assay components, blockers, and the like.

A kit may also include instructions for use in a diagnostic method. Such instructions can be, for example, provided on a device included in the kit, e.g. tools or a device to prepare a biological sample for diagnostic purposes, such as separating a cell and/or protein containing fraction before determining a marker. The kit may conveniently be provided in the storage stable form, such as a commercial kit with a shelf-life of at least 6 months.

Specific diagnostic kits also comprise a solid support comprising a detection molecule or having an immobilized patterned array of detection molecules directed against markers of interest, preferably including a first region containing a first binding reagent directed against a first marker and a second region containing a second binding reagent directed against a second marker.

In particular, a sandwich format can be used. For example, one or more binder is conjugated to a substrate prior to the contacting with a biological sample. The one or more binder may be conjugated to a detectable label to serve as a detection molecule. In other embodiments, the one or more binder is conjugated to a detectable label. In this configuration, the one or more binders may be conjugated to a substrate prior to the contacting with the biological sample to serve as a capture agent. Furthermore, the one or more binder can be conjugated to a substrate prior to the contacting with the biological sample, and/or the one or more binder is conjugated to a detectable label. In such cases, the one or more binder can act as either or both of a capture agent and a detection agent.

The diagnostic kit is specifically provided for use in an immunoassay, wherein the detection molecule is a specific binder that binds to the analyte or marker by an immunoreaction. Such binder may be antibodies or antibody fragments or antibody-like scaffolds binding to a target antigen.

Suitable immunoassays are any of ELISA, CIA, RIA, IRMA, agglutination assay, immunochromatography, dipstick assay and Western-blot.

The term “K. pneumoniae infection” and “K. pneumoniae colonization” is understood in the following way: Klebsiella pneumoniae is a Gram-negative, bacterium that is a member of the family Enterobacteriaceae. It is a ubiquitous bacterium, which can also colonize the human host, typically in the intestines or the upper airways. Being an opportunistic pathogen, from these sites it can invade sterile body sites in case not properly controlled by the immune system. Uncontrolled bacterial replication at these sites will induce inflammation, in a great part, mediated by the endotoxin (i.e. LPS) molecules released from K. pneumoniae. In case of bacteremia, endotoxin molecules may trigger septic shock.

K. pneumoniae colonization means that the subject has a sufficiently high concentration of K. pneumoniae bacteria at a site that they can be detected, yet the bacteria are causing no signs or symptoms. Colonization can persist for a long period of time, with resolution influenced by the immune response to the organism, competition at the site from other organisms and, sometimes, use of antimicrobials.

In general, bacteremias caused by K. pneumoniae may be successfully treated with known conventional antibacterial therapy, such as treatment with antibiotics, steroid and non-steroid inhibitors of inflammation. The present invention provides for a new immunotherapy, employing antibodies specifically recognizing K. pneumoniae, which is optionally combined with anti-bacterial or anti-inflammatory therapy. Exemplary antibiotics used for treating patients with K. pneumoniae infection are aminoglycosides, cephalosporines, aminopenicilines, carbapenems, fluoroquinolons, tygecycline, colistin, etc.

Multi-drug resistant (MDR) K. pneumoniae is particularly understood as those strains demonstrating resistance to three or more classes of antibiotics, e.g. the following agents/groups: penicillins, cephalosporins, carbapenems, aminoglycosides, tetracyclines, fluoroquinolones, nitrofurantoin, trimethoprim (and its combinations), fosfomycin, polymixins, chloramphenicol, azthreonam, or tigecycline.

With the recent emergence of antibiotic-resistant strains, treating bacteremias of this nature has become significantly more difficult. Patients who develop K. pneumoniae disease have longer hospital and ICU stays, high mortality, and greater health care costs than patients without K. pneumoniae disease. Patient care may be improved and nosocomial infections may be reduced by preventing, rather than treating, K. pneumoniae disease prophylaxis when a patient is heavily colonized by K. pneumoniae.

K. pneumoniae disease is specifically understood as a disease caused by K. pneumoniae infection. Such diseases include local and systemic disease. Severe cases of disease are e.g. primary and secondary bacteremia, pneumonia, urinary tract infection, liver abscess, peritonitis, or meningitis.

The term “recombinant” as used herein shall mean “being prepared by or the result of genetic engineering”. A recombinant host specifically comprises an expression vector or cloning vector, or it has been genetically engineered to contain a recombinant nucleic acid sequence, in particular employing nucleotide sequence foreign to the host. A recombinant protein is produced by expressing a respective recombinant nucleic acid in a host. The term “recombinant antibody”, as used herein, includes antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library or library of antigen-binding sequences of an antibody, and (d) antibodies prepared, expressed, created or isolated by any other means that involve splicing of human immunoglobulin gene sequences to other DNA sequences. Such recombinant antibodies comprise antibodies engineered to include rearrangements and mutations which occur, for example, during antibody maturation. In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual, Cold Spring Harbor, (1982).

Selective binding can be further improved by recombinant antibody optimization methods known in the art. For example, certain regions of the variable regions of the immunoglobulin chains described herein may be subjected to one or more optimization strategies, including light chain shuffling, destinational mutagenesis, CDR amalgamation, and directed mutagenesis of selected CDR and/or framework regions.

The term “subject” as used herein shall refer to a warm-blooded mammalian, particularly a human being or a non-human animal. K. pneumoniae is a critically important human pathogen that is also an emerging concern in veterinary medicine. It is present in a wide range of non-human animal species. Thus, the term “subject” may also particularly refer to animals including dogs, cats, rabbits, horses, cattle, pigs and poultry. In particular the medical use of the invention or the respective method of treatment applies to a subject in need of prophylaxis or treatment of a disease condition associated with a K. pneumoniae infection. The subject may be a patient at risk of a K. pneumoniae infection or suffering from disease, including early stage or late stage disease. The term “patient” includes human and other mammalian subjects that receive either prophylactic or therapeutic treatment. The term “treatment” is thus meant to include both prophylactic and therapeutic treatment.

A subject is e.g. treated for prophylaxis or therapy of K. pneumoniae disease conditions. In particular, the subject is treated, which is either at risk of infection or developing such disease or disease recurrence, or a subject that is suffering from such infection and/or disease associated with such infection.

Specifically the term “prophylaxis” refers to preventive measures which is intended to encompass prevention of the onset of pathogenesis or prophylactic measures to reduce the risk of pathogenesis.

Specifically, the treatment may be by interfering with the pathogenesis of K. pneumoniae as causal agent of the condition.

The term “substantially pure” or “purified” as used herein shall refer to a preparation comprising at least 50% (w/w), preferably at least 60%, 70%, 80%, 90% or 95% of a compound, such as a nucleic acid molecule or an antibody. Purity is measured by methods appropriate for the compound (e.g. chromatographic methods, polyacrylamide gel electrophoresis, HPLC analysis, and the like).

The term “therapeutically effective amount”, used herein interchangeably with any of the terms “effective amount” or “sufficient amount” of a compound, e.g. an antibody of the present invention, is a quantity or activity sufficient to, when administered to the subject effect beneficial or desired results, including clinical results, and, as such, an effective amount or synonym thereof depends upon the context in which it is being applied.

An effective amount is intended to mean that amount of a compound that is sufficient to treat, prevent or inhibit such diseases or disorder. In the context of disease, therapeutically effective amounts of the antibody as described herein are specifically used to treat, modulate, attenuate, reverse, or affect a disease or condition that benefits from an inhibition of K. pneumoniae pathogenesis, for example, adhesion and colonization of mucosal surfaces, uncontrolled replication within sterile body sites, and toxicity of host cells by bacterial products.

The amount of the compound that will correspond to such an effective amount will vary depending on various factors, such as the given drug or compound, the pharmaceutical formulation, the route of administration, the type of disease or disorder, the identity of the subject or host being treated, and the like, but can nevertheless be routinely determined by one skilled in the art.

A therapeutically effective amount of the antibody as described herein, such as provided to a human patient in need thereof, may specifically be in the range of 0.5-50 mg/kg, preferably 5-40 mg/kg, even more preferred up to 20 mg/kg, up to 10 mg/kg, up to 5 mg/kg, though higher doses may be indicated e.g. for treating acute disease conditions. The dose can be much lower if a highly potent antibody is used. In such case, the effective amount may be in the range of 0.005 to 5 mg/kg, preferably 0.05 to 1 mg/kg, or at least 0.005 mg/kg, or at least 0.05 mg/kg, and less than 10 mg/kg or less than 1 mg/kg.

Moreover, a treatment or prevention regime of a subject with a therapeutically effective amount of the antibody of the present invention may consist of a single administration, or alternatively comprise a series of applications. For example, the antibody may be administered at least once a year, at least once a half-year or at least once a month. However, in another embodiment, the antibody may be administered to the subject from about one time per week to about a daily administration for a given treatment. The length of the treatment period depends on a variety of factors, such as the severity of the disease, either acute or chronic disease, the age of the patient, the concentration and the activity of the antibody format. It will also be appreciated that the effective dosage used for the treatment or prophylaxis may increase or decrease over the course of a particular treatment or prophylaxis regime. Changes in dosage may result and become apparent by standard diagnostic assays known in the art. In some instances, chronic administration may be required.

Doses for prophylactic treatment are typically in the lower range (e.g. at least 0.005 mg/kg and less than 1 mg/kg), and specifically administered once, e.g. when a subject is identified as being immunocompromised or immunosuppressed and/or at risk of getting in contact with K. pneumoniae, or by a long-term treatment schedule, e.g. at least 2, 3, 4, 5, 6, 7, 8, 9, 10, 11, or 12 doses annually or half-annually. Doses for therapeutic treatment are typically administered in the acute or chronic phase of disease and typically in the higher range (e.g. at least 0.05 or 0.5 mg/kg and less than 10 mg/kg), and specifically administered until cure of the disease, by one or more administrations, e.g. in regular intervals, such as at least 1, 2, 3, or 4 administrations daily, or at least 1, 2, 3, 4, 5, or 6 administrations weekly, or at least 1, 2, 3, or 4 administrations monthly.

Aiming to develop monoclonal antibodies for the prevention and treatment of infections caused by Klebsiella strains, the molecular target of specific mAbs suitably is the LPS O-antigen, which shows limited heterogeneity in Klebsiella. Such O-side chain is considered immunorelevant because not fully masked by bulky capsular polysaccharide.

Once antibodies with the desired binding properties are identified, such antibodies, including antibody fragments can be produced by methods well-known in the art, including, for example, hybridoma techniques or recombinant DNA technology. Recombinant monoclonal antibodies can, for example, be produced by isolating the DNA encoding the required antibody chains and transfecting a recombinant host cell with the coding sequences for expression, using well known recombinant expression vectors, e.g. the plasmids of the invention or expression cassette(s) comprising the nucleotide sequences encoding the antibody sequences. Recombinant host cells can be prokaryotic and eukaryotic cells, such as those described above.

According to a specific aspect, the nucleotide sequence may be used for genetic manipulation to humanize the antibody or to improve the affinity, or other characteristics of the antibody. For example, the constant region may be engineered to more nearly resemble human constant regions to avoid immune response, if the antibody is used in clinical trials and treatments in humans. It may be desirable to genetically manipulate the antibody sequence to obtain greater affinity to the gal-II target and greater efficacy against Klebsiella pneumoniae. It will be apparent to one of skill in the art that one or more polynucleotide changes can be made to the antibody and still maintain its binding ability to the target O1 antigen.

The production of antibody molecules, by various means, is generally well understood. U.S. Pat. No. 6,331,415 (Cabilly et al.), for example, describes a method for the recombinant production of antibodies where the heavy and light chains are expressed simultaneously from a single vector or from two separate vectors in a single cell. Wibbenmeyer et al., (1999, Biochim Biophys Acta 1430(2):191-202) and Lee and Kwak (2003, J. Biotechnology 101: 189-198) describe the production of monoclonal antibodies from separately produced heavy and light chains, using plasmids expressed in separate cultures of host cells. Various other techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

If desired, any of the exemplified antibodies, may be sequenced and the polynucleotide sequence may then be cloned into a vector for expression or propagation. The sequence encoding the antibody may be maintained in vector in a host cell and the host cell can then be expanded and frozen for future use. Production of recombinant monoclonal antibodies in cell culture can be carried out through cloning of antibody genes from B cells by means known in the art.

In another aspect, the invention provides an isolated nucleic acid comprising a sequence that codes for production of the recombinant antibody of the present invention.

An antibody encoding nucleic acid can have any suitable characteristics and comprise any suitable features or combinations thereof. Thus, for example, an antibody encoding nucleic acid may be in the form of DNA, RNA, or a hybrid thereof, and may include non-naturally-occurring bases, a modified backbone, e.g., a phosphorothioate backbone that promotes stability of the nucleic acid, or both. The nucleic acid advantageously may be incorporated in an expression cassette, vector or plasmid of the invention, comprising features that promote desired expression, replication, and/or selection in target host cell(s). Examples of such features include an origin of replication component, a selection gene component, a promoter component, an enhancer element component, a polyadenylation sequence component, a termination component, and the like, numerous suitable examples of which are known.

The present disclosure further provides the recombinant DNA constructs comprising one or more of the nucleotide sequences described herein. These recombinant constructs are used in connection with a vector, such as a plasmid, phagemid, phage or viral vector, into which a DNA molecule encoding any disclosed antibody is inserted.

Monoclonal antibodies are produced using any method that produces antibody molecules by cell lines in culture, e.g. cultivating recombinant eukaryotic (mammalian or insect) or prokaryotic (bacterial) host cells. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63).

Antibodies of the present invention may be identified or obtained employing a hybridoma method. In such method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

MAbs may then be purified from hybridoma supernatants for further testing for its specific binding of the gal-II antigen, and engineering of antibodies, e.g. for different diagnostic or therapeutic purposes.

Gal-II specific antibodies, in some instances, emerge through screening against the single gal-II antigen. To increase the likelihood of isolating differentially binding clones one would apply multiple selective pressures by processively screening against the different antigens.

Screening methods for identifying antibodies with the desired selective binding properties may be done by display technologies using a library displaying antibody sequences or antigen-binding sequences thereof (e.g. using phage, bacterial, yeast or mammalian cells; or in vitro display systems translating nucleic acid information into respective (poly)peptides). Reactivity can be assessed based on ELISA, Western blotting or surface staining with flow cytometry, e.g. using standard assays.

Isolated antigen(s) may e.g. be used for selecting antibodies from an antibody library, e.g. a yeast-displayed antibody library.

For example, the invention specifically provides for gal-II specific antibodies, which are obtained by a process to identify antibodies with specificities to bind the gal-II antigen, e.g. by a specific discovery selection scheme. Accordingly, an antibody library including antibodies showing reactivity with the gal-II target, may be selected for reactivity with the target.

The invention moreover provides pharmaceutical compositions which comprise an antibody as described herein and a pharmaceutically acceptable carrier or excipient. These pharmaceutical compositions can be administered in accordance with the present invention as a bolus injection or infusion or by continuous infusion. Pharmaceutical carriers suitable for facilitating such means of administration are well known in the art.

Pharmaceutically acceptable carriers generally include any and all suitable solvents, dispersion media, coatings, antibacterial and antifungal agents, isotonic and absorption delaying agents, and the like that are physiologically compatible with an antibody or related composition or combination provided by the invention. Further examples of pharmaceutically acceptable carriers include sterile water, saline, phosphate buffered saline, dextrose, glycerol, ethanol, and the like, as well as combinations of any thereof.

In one such aspect, an antibody can be combined with one or more carriers appropriate a desired route of administration, antibodies may be, e.g. admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tableted or encapsulated for conventional administration. Alternatively, an antibody may be dissolved in saline, water, polyethylene glycol, propylene glycol, carboxymethyl cellulose colloidal solutions, ethanol, corn oil, peanut oil, cotton-seed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well known in the art.

Additional pharmaceutically acceptable carriers are known in the art and described in, e.g. REMINGTON'S PHARMACEUTICAL SCIENCES. Liquid formulations can be solutions, emulsions or suspensions and can include excipients such as suspending agents, solubilizers, surfactants, preservatives, and chelating agents.

Pharmaceutical compositions are contemplated wherein an antibody of the present invention and one or more therapeutically active agents are formulated. Stable formulations of the antibody of the present invention are prepared for storage by mixing said immunoglobulin having the desired degree of purity with optional pharmaceutically acceptable carriers, excipients or stabilizers, in the form of lyophilized formulations or aqueous solutions. The formulations to be used for in vivo administration are specifically sterile, preferably in the form of a sterile aqueous solution. This is readily accomplished by filtration through sterile filtration membranes or other methods. The antibody and other therapeutically active agents disclosed herein may also be formulated as immunoliposomes, and/or entrapped in microcapsules.

Administration of the pharmaceutical composition comprising an antibody of the present invention, may be done in a variety of ways, including orally, subcutaneously, intravenously, intranasally, intraotically, transdermally, mucosal, topically, e.g., gels, salves, lotions, creams, etc., intraperitoneally, intramuscularly, intrapulmonary, e.g. employing inhalable technology or pulmonary delivery systems, vaginally, parenterally, rectally, or intraocularly.

Exemplary formulations as used for parenteral administration include those suitable for subcutaneous, intramuscular or intravenous injection as, for example, a sterile solution, emulsion or suspension.

In one embodiment, the antibody of the present invention is the only therapeutically active agent administered to a subject, e.g. as a disease modifying or preventing monotherapy.

In another embodiment, the antibody of the present invention is combined with further antibodies in a cocktail, e.g. combined in a mixture or kit of parts, to target Klebsiella pneumoniae, such that the cocktail contains more than one therapeutically active agents administered to a subject, e.g. as a disease modifying or preventing combination therapy.

Further, the antibody of the present invention may be administered in combination with one or more other therapeutic or prophylactic agents, including but not limited to standard treatment, e.g. antibiotics, steroid and non-steroid inhibitors of inflammation, and/or other antibody based therapy, e.g. employing anti-bacterial or anti-inflammatory agents.

A combination therapy is particularly employing a standard regimen, e.g. as used for treating infection by Klebsiella pneumoniae. This may include antibiotics, e.g., tygecycline, colistin, polymixin B, and beta lactams combined with non-beta lactam inhibitors.

In a combination therapy, the antibody may be administered as a mixture, or concomitantly with one or more other therapeutic regimens, e.g. either before, simultaneously or after concomitant therapy.

The biological properties of the antibody or the respective pharmaceutical preparations of the invention may be characterized ex vivo in cell, tissue, and whole organism experiments. As is known in the art, drugs are often tested in vivo in animals, including but not limited to mice, rats, rabbits, dogs, cats, pigs, and monkeys, in order to measure a drug's efficacy for treatment against a disease or disease model, or to measure a drug's pharmacokinetics, pharmacodynamics, toxicity, and other properties. The animals may be referred to as disease models. Therapeutics are often tested in mice, including but not limited to nude mice, SCID mice, xenograft mice, and transgenic mice (including knockins and knockouts). Such experimentation may provide meaningful data for determination of the potential of the antibody to be used as a therapeutic or as a prophylactic with the appropriate half-life, effector function, bactericidal activity and/or immune response upon active or passive immunotherapy. Any organism, preferably mammals, may be used for testing. For example because of their genetic similarity to humans, primates, monkeys can be suitable therapeutic models, and thus may be used to test the efficacy, toxicity, pharmacokinetics, pharmacodynamics, half-life, or other property of the subject agent or composition. Tests in humans are ultimately required for approval as drugs, and thus of course these experiments are contemplated. Thus, the antibody and respective pharmaceutical compositions of the present invention may be tested in humans to determine their therapeutic or prophylactic efficacy, toxicity, immunogenicity, pharmacokinetics, and/or other clinical properties.

In specific cases the patient is an immunocompromised patient. Some immunocompromised patients may suffer from a primary immunodeficiency or a secondary immunodeficiency. Some immunocompromised patients are being or have been treated with an immunosuppressive therapy or with a chemotherapeutic agent. Some immunocompromised patients are transplant patient.

Immunocompromised patients likely suffer from a phagocytic disorder, such as characterized by a lower phagycytic number and/or impaired function.

The following disorders can cause impaired or lost phagocytotic activities:

Primary immunodeficiency of phagocytes (4):

1. Chronic neutropenia:

a. Cyclic neutropenia

b. Severe congenital neutropenia

c. Shwachman-Diamond syndrome

2. Leukocyte adhesion deficiency

a. Type 1

b. Type 2

c. Rac 2 deficiency

3. Defects of signaling

a. Interferon-γ and interleukin-12 defects

4. Defects of intracellular killing

a. Chronic granulomatous disease of childhood

b. Myeloperoxidase deficiency

c. Chediak-Higashi syndrome

d. Neutrophil-specific granule deficiency

Secondary immunodeficiency of phagocytes (5):

1. Neutropenia/granulocytopenia: reduced number of blood neutrophils/granulocytes (<1500 cells/ml)

a. Bone marrow diseases (tumor infiltration, aplastic anaemia, hematologic malignancy, granulomatous disease, irradiation, myelofibrosis)

b. Immune mediated neutropenia (drugs acting as hapten, autoimmune diseases)

c. Infections (bacterial sepsis, malaria, toxoplasmosis, viral infections, like EBV, CMV, Influenza)

d. Nutritional deficiency (malnutrition, B-12 deficiency)

e. Drugs, chemicals (macrolids, procainamides, phenotiazid, sulfonamides, chloramphenicol, aminopyrine, anti-thyroid drugs, like thiouracil, methimazol, thiocyanate, heavy metals) (6)

f. Chemotherapy, immunosuppression (treatment of autoimmune diseases, after transplantation)

2. Phagocyte function/chemotaxis disorder or decreased ability to upregulate production of phagocytes (7)

a. Neonates (Under conditions of stress, neonatal PMNs do not function with normal phagocytic and microbicidal activities. PMNs isolated from the blood of term neonates display diminished chemotactic and adhesion capacities. (8)

b. Elderly (Decreased phagocytic ability, cytotoxicity, enzyme release, reduced adhesion (9)

c. Diabetes mellitus (lower killing by PMNs, monocyte/macrophage dysfunction (10), renal failure and cirrhosis

d. Trisomy 21

e. Surgery, trauma

f. Corticosteroids

g. HIV

To identify patients with impaired phagocyte number and function, any suitable technique known by persons skilled in the art can be applied. These include but are not limited to complete blood count, differential white blood cell count, peripheral smear, measurement of adherence, chemotaxis, phagocytosis, intracellular killing of phagocytes, assays to measure specific neutrophil enzymes or detect autoantibodies against neutrophils.

Therefore, the invention particularly provides antibodies that show direct bactericidal activity, i.e. not dependent on cellular immune status of the host. Based on this novel mode of action of anti-galactan-II mAbs, such mAbs could be used in the immunocompromised patient population as add-on or standalone therapeutic in case of invasive infections by K. pneumoniae O1. The antibodies could offer a new preventive measure at individuals being at risk of acquiring an immunocompromised condition with decrease phagocytic function (cancer patients before chemotherapy or radiation therapy, patients undergoing immunosuppressive therapy) or at patients on clinical wards affected by K. pneumoniae outbreaks.

The present invention is further illustrated by the following examples without being limited thereto.

### EXAMPLES

### Example 1: Development of O1-Specific Humanized mAbs

Murine mAbs against the O1 carbohydrate antigen were developed by standard hybridoma technology. Briefly, mice were immunized 4-times with sublethal doses of live bacteria. Following fusion of splenocytes the specific hybridoma clones were selected using extracted O1 LPS (immunoblots) or derived biotinylated polysaccharide antigens (ELISA) as well as flow cytometry with whole bacterial cells. The specific mAbs were expressed as murine-human chimeric antibodies (mouse variable regions fused to human IgG1 constant heavy and kappa constant light chain regions). The most efficacious mAbs were subjected to humanization, where murine framework regions were replaced by corresponding human regions, leaving exclusively the hypervariable CDR regions as murine sequences.

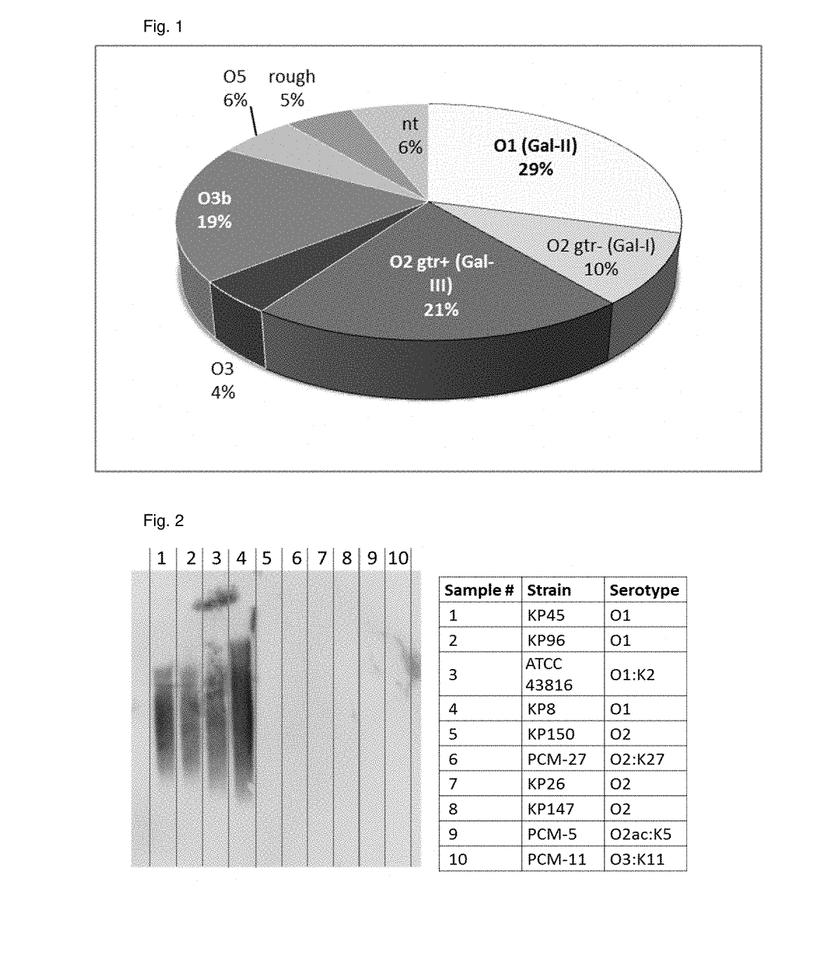

Binding specificity of O1 mAbs was confirmed with immunoblots using separated (SDS-PAGE) extracted purified LPS molecules blotted onto PVDF membranes. Reactivity pattern of the O1 mAbs is exemplified by mAb 8E9 (FIG. 2). The mAb was reacted for 1 h at 1 μg/ml concentration. Blot was developed using HRP-labelled anti-mouse IgG secondary antibodies and luminography. The O1 specific mAb stained the long molecular weight LPS fraction suggesting a specific binding to galactan-II epitopes. The lack of binding to O2 type LPS molecules confirmed this specificity (since galactan-I, the other O-antigen repeating unit of serotype O1, is shared by O1 and O2 LPS molecules).

### Example 2: O1 mAbs are Capable to Bind to the Bacterial Surface Irrespective of the Capsular Type

For the intended bactericidal activity it is indispensable that mAbs bind to the surface and trigger Fc-dependent effector functions. K. pneumoniae, however, shields its surface molecules by abundant capsular polysaccharide (CPS) that shows high structural variability. Therefore, it was considered important to show that the discovered O1-specific mAbs can efficiently bind to the bacterial surface in the presence of different CPS coats.

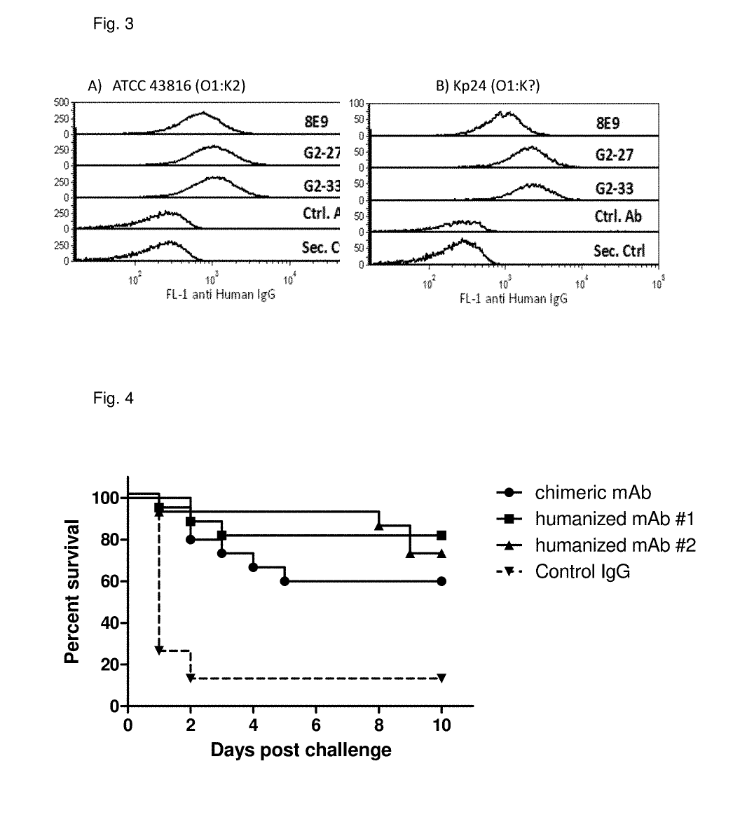

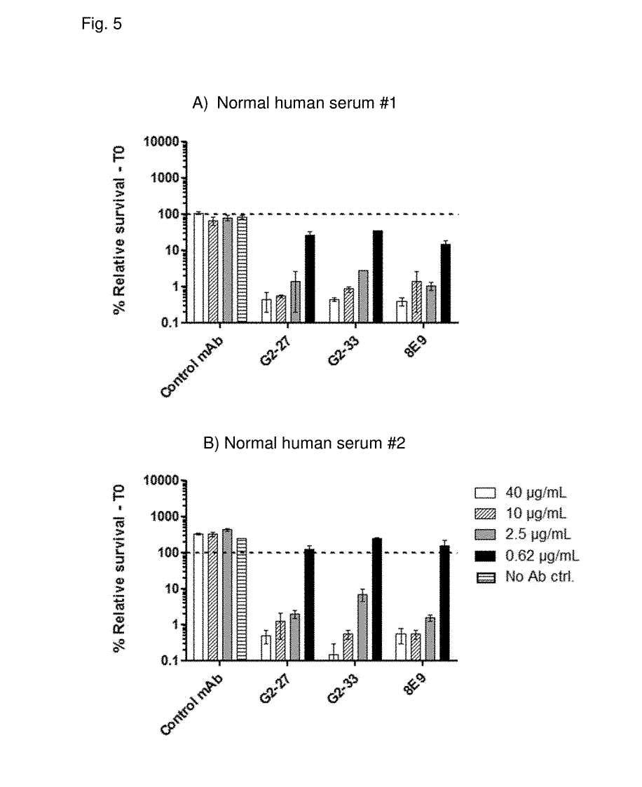

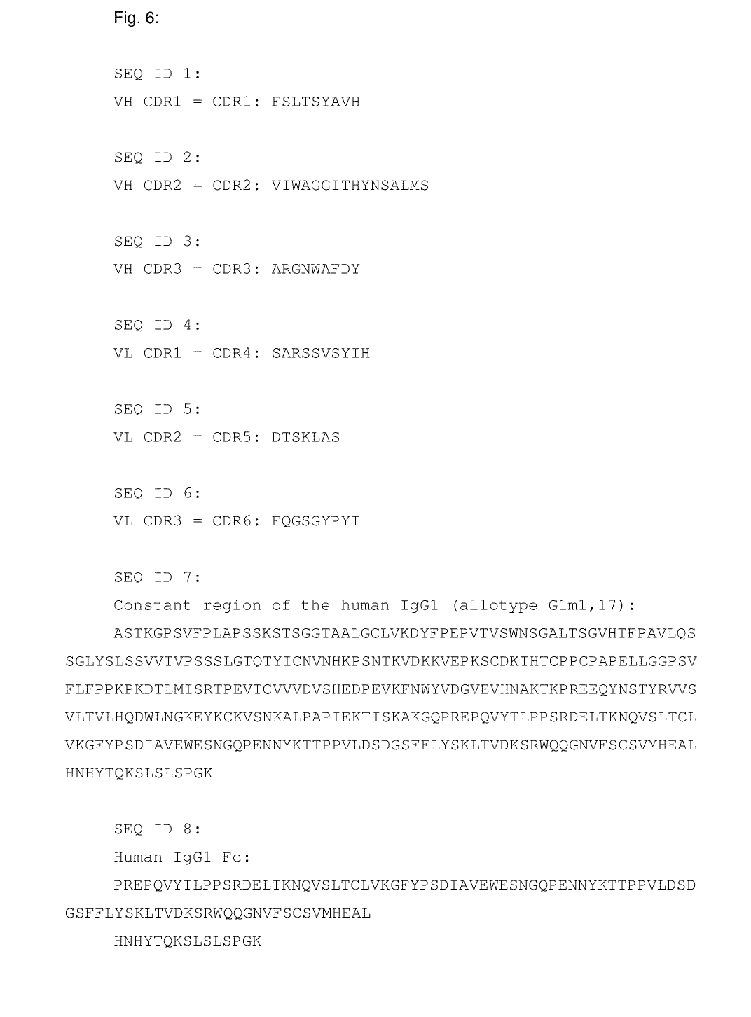

Mid-log cultures of K. pneumoniae O1 strains expressing either K2 (ATCC 43816) or a genetically confirmed non-K2 (clinical isolate) CPS were stained with 40 μg/ml of humanized or chimeric O1-specific mAbs and subsequently with a secondary anti-human IgG labelled with Alexa 488. Fluorescence of bacteria was measured by flow cytometry. As depicted in FIG. 3 O1-specific mAbs showed strong surface staining on both strains suggesting that different CPS molecules do not hinder accessibility of O1 epitopes for mAb binding.

### Example 3: Protective Capacity in a Murine Model of Bacteremia

Protective efficacy of purified humanized mAbs as well as their parental chimeric mAb was tested in a murine model of K. pneumoniae bacteremia (FIG. 4). Mice were immunized with 50 ug of mAb intraperitoneally 24 h prior to a lethal intravenous challenge (5×10⁶ CFU) with K. pneumoniae O1:K2 strain ATCC 43816. Lethality was monitored daily for 10 days. Both the chimeric mAbs as well as the humanized derivatives showed significant protection over the control group receiving an isotype matched irrelevant mAb at the same dose.

### Example 4: Phagocyte-Independent Bactericidal Activity

Given that K. pneumoniae strains tend to infect immunocompromised patients with limited phagocytic capacity, we considered important to find mAbs with direct bactericidal activity. Phagocyte-independent complement mediated bactericidal activity of the mAbs was tested in a so-called serum bactericidal assay (SBA). O1-specific humanized mAbs G2-27 and G2-33 as well as their parental murine-human chimeric mAb elicit dose-dependent complement-mediated bacterial killing in both serum samples tested. No bactericidal activity was observed when using an isotype matched mAb with irrelevant specificity or upon heat-inactivation (56 C for 30 min) of the sera used (not shown) corroborating an antibody dependent complement mediated killing.

This effect is not obvious, since a reported galactan-II-specific murine IgG2b—although it was capable of inducing complement-dependent opsonophagocytic killing—lacked complement mediated killing (i.e. no bactericidal activity was observed in the absence of phagocytes (1).

### REFERENCE LIST

(1) Held T K, Jendrike N R, Rukavina T, Podschun R, Trautmann M. Binding to and opsonophagocytic activity of O-antigen-specific monoclonal antibodies against encapsulated and nonencapsulated Klebsiella pneumoniae serotype O1 strains. Infect Immun 2000 May; 68(5):2402-9.
(2) Rukavina T, Ticac B, Susa M, et al. Protective effect of antilipopolysaccharide monoclonal antibody in experimental Klebsiella infection. Infect Immun 1997 May; 65(5):1754-60.
(3) Trautmann M, Ruhnke M, Rukavina T, et al. O-antigen seroepidemiology of Klebsiella clinical isolates and implications for immunoprophylaxis of Klebsiella infections. Clin Diagn Lab Immunol 1997 September; 4(5):550-5.
(4) Lekstrom-Himes J A, Gallin J I. Immunodeficiency diseases caused by defects in phagocytes. N Engl J Med 2000 Dec. 7; 343(23):1703-14.
(5) Chinen J, Shearer W T. Secondary immunodeficiencies, including HIV infection. J Allergy Clin Immunol 2010 February; 125(2 Suppl 2):S195-S203.
(6) Bhatt V, Saleem A. Review: Drug-induced neutropenia-pathophysiology, clinical features, and management. Ann Clin Lab Sci 2004; 34(2):131-7.
(7) Engelich G, Wright D G, Hartshorn K L. Acquired disorders of phagocyte function complicating medical and surgical illnesses. Clin Infect Dis 2001 Dec. 15; 33(12):2040-8.
(8) Clapp D W. Developmental regulation of the immune system. Semin Perinatol 2006 April; 30(2):69-72.
(9) Wenisch C, Patruta S, Daxbock F, Krause R, Horl W. Effect of age on human neutrophil function. J Leukoc Biol 2000 January; 67(1):40-5.
(10) Geerlings S E, Hoepelman A I. Immune dysfunction in patients with diabetes mellitus (DM). FEMS Immunol Med Microbiol 1999 December; 26(3-4):259-65.
(11) Whitfield C, Richards J C, Perry M B, Clarke B R, MacLean LL. Expression of two structurally distinct D-galactan O antigens in the lipopolysaccharide of Klebsiella pneumoniae serotype O1. J Bacteriol 1991 February; 173(4):1420-31.
(12) Kol O, Wieruszeski J M, Strecker G, Fournet B, Zalisz R, Smets P. Structure of the O-specific polysaccharide chain of Klebsiella pneumoniae O1 K2 (NCTC 5055) lipopolysaccharide. A complementary elucidation. Carbohydr Res 1992 Dec. 15; 236:339-44.
(13) Vinogradov et al. 2002, J. Biol. Chem. 277(28): 25070-25081.
(14) Hsieh et al. 2014, Frontiers in Microbiology 5, Article 608:1-14.
(15) Kubota et al. 2009, Cancer sci. 100:1566-1572.

1-19. (canceled)

20. A human or humanized monoclonal IgG antibody (mAb) specifically recognizing D-galactan-II of Klebsiella pneumoniae serotype O1 which is characterized by a bactericidal CDC activity.

21. The antibody of claim 20, wherein the antibody is a human or humanized mAb.

22. The antibody of claim 20 which comprises
A
the antigen-binding site characterized by the following CDR sequences:
a) CDR1 consisting of the amino acid sequence of SEQ ID 1; and
b) CDR2 consisting of the amino acid sequence of SEQ ID 2; and
c) CDR3 consisting of the amino acid sequence of SEQ ID 3; and
d) CDR4 consisting of the amino acid sequence of SEQ ID 4; and
e) CDR5 consisting of the amino acid sequence of SEQ ID 5; and
f) CDR6 consisting of the amino acid sequence of SEQ ID 6;
or
B
a functional variant of the antigen-binding site as defined in A, wherein the functional variant comprises at least one point mutation in any one or more of the CDR sequences, and further wherein
i. the functional variant has a specificity to bind the gal-II epitope; and/or
ii. the functional variant is a human, humanized, or an affinity matured variant of the antigen-binding site.

23. The antibody of claim 20, which is a full-length monoclonal antibody, an antibody fragment thereof comprising at least one antibody domain construct incorporating the antigen-binding site and the Fc region, or a fusion protein comprising at least said antibody fragment fused to a heterologous peptide or polypeptide.

24. The antibody of claim 20, comprising the Fc of a human IgG1 or IgG3, preferably any human IgG1 or IgG3 allotype, preferably the constant region of human IgG1 allotype G1m1,17 identified by the amino acid sequence SEQ ID 7, or the Fc part thereof, or a functional variant thereof, comprising a C1q binding site.

25. The antibody of claim 20, which has an affinity to bind the O1 antigen with a Kd of less than 10⁻⁶M, less than 10⁻⁷M or less than 10⁻⁸M.

26. An artificial monoclonal antibody composition comprising the antibody of claim 20.

27. A pharmaceutical preparation comprising the antibody of claim 20, and, optionally, a pharmaceutically acceptable carrier or excipient.

28. The method of claim 33, for treatment or prophylaxis of any of primary and secondary bacteremia, pneumonia, urinary tract infection, liver abscess, peritonitis, or meningitis.

29. The method according to claim 33, wherein the subject is an immunocompromised or immunosuppressed patient, or a contact thereof.

30. The method according to claim 33, wherein the subject is of a host group characterized by an impaired phagocyte number and/or function, which host group is any of
a) patients suffering from inherited or acquired primary or secondary immunodeficiency;
b) patients selected from the group consisting of neonates younger than x months of age, elderly patients older than 65 years of age, patients suffering from Diabetes mellitus, renal failure, cirrhosis, Trisomie 21, trauma, or HIV, or patients who have undergone surgical interventions or systemic treatment with corticosteroids; or
c) patients admitted to hospital or hospital personnel with a risk of contracting infection upon exposure to a patient suffering from K. pneumoniae disease.

31. The method according to claim 33, to prevent nosocomial or iatrogenic outbreaks of K. pneumoniae disease.

32. The method according to claim 33, wherein the antibody is administered in combination with an antibiotic drug.

33. A method of treating a subject at risk of or suffering from Klebsiella pneumoniae infection or colonization comprising administering to the subject an effective amount of the antibody of claim 20 to limit the infection in the subject or to ameliorate a disease condition resulting from said infection.

34. A diagnostic preparation in a composition or a kit of parts, comprising
a) the antibody of claim 20;
b) a further diagnostic reagent; and
c) optionally a solid phase to immobilize at least one of the antibody and the diagnostic reagent.

35. A method of diagnosing Klebsiella pneumoniae O1 infection or colonization in a subject caused by a Klebsiella pneumoniae O1 strain, comprising
a) providing an antibody according to claim 20, and
b) detecting if the antibody specifically immunoreacts with the galactan-II epitope in a biological sample of the subject to be tested, thereby diagnosing Klebsiella pneumoniae O1 infection or colonization.

36. A nucleic acid encoding the antibody of claim 20.

37. A recombinant host cell comprising the nucleic acid of claim 36.

38. A method of producing the antibody of claim 20, wherein a recombinant host cell capable of expressing the antibody is cultivated or maintained under conditions to produce said antibody.

39. The pharmaceutical preparation of claim 27, comprising a parenteral or mucosal formulation.

40. The diagnostic preparation of claim 34, wherein the further diagnostic reagent is a diagnostic label or a reagent specifically reacting with the antibody and/or the reaction product of the antibody binding to its antigen.
